(19) United States
(12) Patent Application Publication
(10) Pub. No.: US 2005/0176724 A1
(43) Pub. Date: Aug. 11, 2005

(54) Piperidine and piperazine derivatives possessing affinity at 5ht-1 type receptors

(76) Inventors: Sean F. Flynn, Essex (GB); Paul W. Smith, Essex (GB); Kevin M. Thewlis, Essex (GB); Simon E. Ward, Essex (GB)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 10/504,114

(22) PCT Filed: Feb. 17, 2003
(86) PCT No.: PCT/EP03/01710

(30) Foreign Application Priority Data
Feb. 18, 2002 (GB) 0203778.6

(51) Int. Cl. A61K 31/517; A61K 31/496; C07D 43/02

(52) U.S. Cl. 514/252.17; 514/253.06; 544/284; 544/363

(57) ABSTRACT

Compounds of formula (I) and pharmaceutically acceptable salts thereof are disclosed: 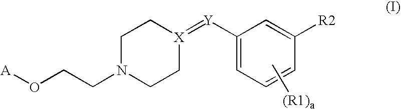
wherein A is optionally substituted phenyl, indolyl, quinolinyl, quinazolinyl, indazolyl or isoquinolinyl; X is carbon, Y is CH and ═ is a double bond; or X is CH, Y is CH₂ or oxygen and ═ is a single bond; or X is nitrogen, Y is CH₂ and ═ is a single bond; R1 is halogen, cyano, nitro, C₁₋₆alkyl, haloC₁₋₆alkyl, C₁₋₆alkoxy, C₃₋₇heterocyclylC₁₋₆alkyl, C₃₋₇heterocyclyl C₁₋₆alkoxy; a is 0, 1, 2, 3 or 4; R2 is either: halogen, —CN, an optionally substituted C₃₋₇cycloalkyl, an optionally substituted aryl or an optionally substituted C-linked 3-7 membered heterocyclic group; or a group -(Z)b-B wherein: (i) Z is oxygen, CH₂, C═O, SO₂ or C═N—OR3 wherein R3 is hydrogen or C₁₋₆alkyl; b is 1, 2, or 3; and B is hydrogen, C₁₋₆alkyl, C₃₋₇cycloalkyl, C₁₋₆alkoxy or NR4R5 wherein R4 and R5 are independently hydrogen, C₁₋₆alkyl, C₃₋₇cycloalkyl, C₁₋₆alkanoyl, fluoroC₁₋₆alkanoyl, C₁₋₆alkylsulfonyl, fluoroC₁₋₆alkylsulfonyl, carbamoyl or C₁₋₆alkylcarbamoyl, or R4 and R5, together with the nitrogen atom to which they are attached, form part of an optionally substituted 3 to 7 membered heterocyclic group; or (ii) Z is oxygen, CH or CH₂, b is 1, and B forms the rest of an aryl or a C₃₋₇heterocyclic group fused to the phenyl ring; excluding 1-[2-[2-(phenylmethyl)phenoxy]ethyl]-4-[(2,3,4-trimethoxyphenyl)methyl]-piperazine and pharmaceutically acceptable salts thereof, and N-[4-[2-[4-[(3,4-dimethoxyphenyl)methyl]-1-piperazinyl]ethoxy]phenyl]-ethanesulfonamide and pharmaceutically acceptable salts thereof. Methods of preparing the compounds and uses of the compounds in therapy, in particular for CNS disorders such as depression and anxiety, are also disclosed.

The present invention relates to novel compounds, processes for their preparation, pharmaceutical compositions containing the same and their use as medicaments in the treatment of CNS and other disorders.

WO 90/13539 discloses certain N-substituted piperazine derivatives, such as 1-[2-[2-(phenylmethyl)phenoxy]ethyl]-4-[(2,3,4-trimethoxyphenyl)methyl]-piperazine, which are said to be useful for patients suffering from functional disorder of brains, such as caused by necrosis, cerebral circulation disorder or anoxia.

WO 96/05174 discloses certain amine derivatives, such as N-[4-[2-[4-[(3,4-dimethoxyphenyl)methyl]-1-piperazinyl]ethoxy]phenyl]-methanesulfonamide, which are said to be useful as antiarrhythmic agents.

A novel series of compounds has now been found that possess high affinity for 5-HT₁ type receptors. The present invention therefore provides, in a first aspect, a compound of formula (I) or a pharmaceutically acceptable salt thereof: 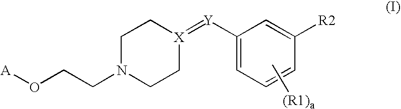
wherein
A is optionally substituted phenyl, indolyl, quinolinyl, quinazolinyl, indazolyl or isoquinolinyl;
X is carbon, Y is CH and ═ is a double bond; or X is CH, Y is CH₂ or oxygen and ═ is a single bond; or X is nitrogen, Y is CH₂ and ═ is a single bond;
R1 is halogen, cyano, nitro, C₁₋₆alkyl, haloC₁₋₆alkyl, C₁₋₆alkoxy, C₃₋₇heterocyclylC₁₋₆alkyl, C₃₋₇heterocyclyl C₁₋₆alkoxy;
a is 0, 1, 2, 3 or 4;
R2 is either:
   halogen, —CN, an optionally substituted C₃₋₇cycloalkyl, an optionally substituted aryl or an optionally substituted C-linked 3-7 membered heterocyclic group; or
   a group -(Z)b-B wherein:
      (i) Z is oxygen, CH₂, C═O, SO₂ or C═N—OR3 wherein R3 is hydrogen or C₁₋₆alkyl; b is 1, 2, or 3; and B is hydrogen, C₁₋₆alkyl, C₃₋₇cycloalkyl, C₁₋₆alkoxy or NR4R5 wherein R4 and R5 are independently hydrogen, C₁₋₆alkyl, C₃₋₇cycloalkyl, C₁₋₆alkanoyl, fluoroC₁₋₆alkanoyl, C₁₋₆alkylsulfonyl, fluoroC₁₋₆alkylsulfonyl, carbamoyl or C₁₋₆alkylcarbamoyl, or R4 and R5, together with the nitrogen atom to which they are attached, form part of an optionally substituted 3 to 7 membered heterocyclic group; or
      (ii) Z is oxygen, CH or CH₂, b is 1, and B forms the rest of an aryl or a C₃₋₇heterocyclic group fused to the phenyl ring;
excluding
1-[2-[2-(phenylmethyl)phenoxy]ethyl]-4-[(2,3,4-trimethoxyphenyl)methyl]-piperazine and pharmaceutically acceptable salts thereof, and
N-[4-[2-[4-[(3,4-dimethoxyphenyl)methyl]-1-piperazinyl]ethoxy]phenyl]-methanesulfonamide and pharmaceutically acceptable salts thereof.

The term “halogen” and its abbreviation “halo” refer to fluorine, chlorine, bromine or iodine.

The term “C₁₋₆alkyl” refers to an alkyl group having from one to six carbon atoms, in all isomeric forms, such as methyl, ethyl, propyl, isopropyl, butyl, isobutyl, sec-butyl, tert-butyl, pentyl, neopentyl, sec-pentyl, n-pentyl, isopentyl, tert-pentyl and hexyl.

The term “haloC₁₋₆alkyl” refers to an alkyl group having one or more substitutions by halogen atoms, such as for example CF₃.

The term “C₁₋₆alkoxy” refers to a straight chain or branched chain alkoxy (or “alkyloxy”) group having from one to six carbon atoms, such as methoxy, ethoxy, propoxy, isopropoxy, butoxy, isobutoxy, sec-butoxy, tert-butoxy, pentoxy, neopentoxy, sec-pentoxy, n-pentoxy, isopentoxy, tert-pentoxy and hexoxy.

The term “C₁₋₆alkanoyl” refers to an alkanoyl group having from 1 to 6 carbon atoms, such as methanoyl (or “formyl”), ethanoyl (or “acetyl”), propanoyl, isopropanoyl, butanoyl, isobutanoyl, sec-butanoyl, pentanoyl, neopentanoyl, sec-pentanoyl, isopentanoyl, tertpentanoyl and hexanoyl.

The term “aryl”, whether alone or as part of another group, is intended, unless otherwise stated, to denote an aromatic carbocyclic or heterocyclic group such as phenyl, naphthyl, pyridyl, pyrimidinyl, pyrazinyl, quinolyl, isoquinolyl, indolyl, isoindolyl or indazolyl, optionally substituted by one or more, preferably 1 to 3, halogen, C₁₋₆alkyl, CF₃, cyano, hydroxy, C₁₋₆alkanoyl, or C₁₋₆alkoxy. Where used herein the term naphthyl, whether alone or as part of another group, is intended, unless otherwise stated, to denote both 1-naphthyl and 2-naphthyl groups.

The terms “C₃₋₇heterocyclyl”, “C₃₋₇heterocyclic group” and “3 to 7 membered heterocyclic group” refer to an optionally substituted saturated or non-saturated ring consisting of a total of 3 to 7 atoms and containing 1, 2 or 3 heteroatoms selected from nitrogen, sulphur or oxygen. Examples of such heterocyclic groups include aziridinyl, azetidinyl, furyl, thienyl, tetrahydrofuryl, tetrahydrothienyl, dioxanyl, pyrrolidinyl, imidazolidinyl, pyrazolidinyl, oxazolidinyl, isothiazolidinyl, thiazolidinyl, pyrrolyl, pyrrolinyl, pyrazolinyl, imidazolyl, pyrazolyl, thiazolyl, piperidyl, piperazinyl, morpholinyl, thiomorpholinyl, azepinyl and azepanyl. These groups may be substituted by one or more, preferably 1 to 3, substituents, which may be the same or different, and which is selected from the following group: halogen, oxo, C₁₋₆alkyl, cyano, CF₃, C₁₋₆alkoxy and C₁₋₆alkanoyl. The substituent(s) may be attached to any available carbon, nitrogen or sulphur atom. Substituents in the heterocycle may form a bridge structure, to form a group such as for example 2-azabicyclo[2.2.2]octyl. Such a bicyclic group may be further substituted by one or more halogen, oxo, C₁₋₆alkyl, cyano, CF₃, C₁₋₆alkoxy or C₁₋₆alkanoyl.

The term “C-linked heterocyclic group” refers to a heterocyclic group which is joined to the rest of the molecule via a carbon atom.

The term “C₃₋₇heterocyclylC₁₋₆alkyl” refers to a C₃₋₇heterocyclyl group which is joined to a C₁₋₆alkyl group, such as pyridylethyl, morpholinylethyl and piperidinylmethyl. Similarly, the term “C₃₋₇heterocyclylC₁₋₆alkoxy” refers to groups such as pyridylethoxy, morpholinylethoxy and piperidinylmethoxy.

The term “C₃₋₇cycloalkyl” refers to a cycloalkyl group consisting of from 3 to 7 carbon atoms, such as cyclopropane, cyclobutane, cyclopentane, cyclohexane and cycloheptane, which is optionally substituted by one or more, preferably 1 to 3, halogen, hydroxy, oxo, C₁₋₆alkyl, cyano, CF₃, C₁₋₆alkoxy or C₁₋₆alkanoyl.

The term “fluoroC₁₋₆alkanoyl” refers to a fluorine-substituted C₁₋₆alkanoyl group such as CF₃CO. The term “fluoroC₁₋₆alkylsulfonyl” refers to a fluorine-substituted C₁₋₆alkylsulfonyl group such as CF₃SO₂.

The term “carbamoyl” refers to the group H₂NCO. The term “C₁₋₆alkylcarbamoyl” refers to a group having the formula (C₁₋₆alkyl)HNCO, such as CH₃NHCO.

The term “oxo” refers to the group “═O”.

When a is two or more, the two or more R1 groups may be the same or different. When b is two or more, the two or more Z groups may be the same or different.

The following compounds are excluded from the scope of the present application:
1-[2-[2-(phenylmethyl)phenoxy]ethyl]4-[(2,3,4trimethoxyphenyl)methyl]-piperazine: 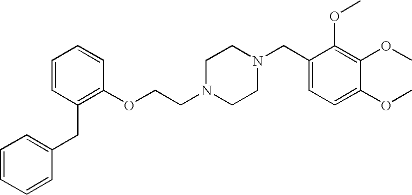
   and pharmaceutically acceptable salts thereof, and
N-[4-[2-[4-[(3,4-dimethoxyphenyl)methyl]-1-piperazinyl]ethoxy]phenyl]-methanesulfonamide: 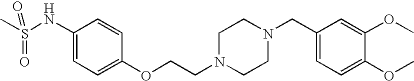
   and pharmaceutically acceptable salts thereof.

A is optionally substituted phenyl, indolyl, quinolinyl, quinazolinyl, indazolyl or isoquinolinyl. These groups may be attached to the oxygen atom at any suitable position. These groups may be substituted by 1 to 4 substituents, which may be the same or different, and which are selected from the following group: halogen, hydroxy, cyano, CF₃, C₁₋₆alkyl, C₁₋₆alkoxy, C₁₋₆alkoxyC₁₋₆alkyl, C₃₋₇cycloalkylC₁₋₆alkoxy, C₁₋₆alkanoyl, C₁₋₆alkylsulfonyl, C₁₋₆alkylsulfinyl, C₁₋₆alkylsulfonyloxy, C₁₋₆alkylsulfonylC₁₋₆alkyl, C₁₋₆alkylsulfonamido, C₁₋₆alkylamido, C₁₋₆alkylsulfonamidoC₁₋₆alkyl and C₁₋₆alkylamidoC₁₋₆alkyl. Preferred optional substituents for A are C₁₋₆alkyl, cyano, CF₃, C₁₋₆alkoxy and C₁₋₆alkanoyl.

Preferably A is quinolinyl or quinazolinyl. Most preferably, A is 5-(2-methyl)quinolinyl or 5-(2-methyl)quinazolinyl.

Preferably Y is CH₂ or CH.

Preferably R1 is fluoro.

Preferably a is 0, 1 or 2.

When R2 is an optionally substituted C-linked 3 to 7 membered heterocyclic group, preferably it is a 6 membered saturated heterocyclic group such as piperidyl.

When R2 is a substituted C₃₋₇cycloalkyl, a substituted aryl or a substituted C-linked 3 to 7 membered heterocyclic group, preferably the number of substituents is 1, 2 or 3, and the substituents are independently selected from: fluorine, chlorine, oxo and C₁₋₆alkyl.

Preferably, when R2 is a group (Z)b-B, and Z is oxygen, f is 1 and P is methyl, such that the group (Z)b-B forms a methoxy group, and d is 1, 2, 3 or 4, then any of the one or more R1 is not methoxy.

Within the group (Z)b-B, when B is NR4R5, preferably R4 and R5 are independently hydrogen, C₁₋₆alkyl (particularly methyl, ethyl or propyl), C₁₋₆alkanoyl, C₁₋₆alkylsulfonyl, haloC₁₋₆alkanoyl or C₁₋₆alkylcarbamoyl. More preferably, one of R4 and R5 is hydrogen or C₁₋₆alkyl (particularly methyl, ethyl or propyl) and the other is C₁₋₆alkanoyl, C₁₋₆alkylsulfonyl, haloC₁₋₆alkanoyl, or C₁₋₆alkylcarbamoyl, or R4 and R5, together with the nitrogen atom to which they are attached, form a saturated 5 or 6 membered heterocyclic group such as pyrrolidinyl, imidazolinyl, isothiazolidinyl, thiazolidinyl, morpholinyl, piperidinyl or piperazinyl, optionally substituted by 1 or 2 substituents selected from halogen, oxo, C₁₋₆alkyl, cyano, CF₃, C₁₋₆alkoxy and C₁₋₆ alkanoyl.

When Z is oxygen, CH or CH₂, the group (Z)b-B may form an aryl or a C3-7heterocyclic group which is fused to the phenyl ring at one of the two carbon atoms which are ortho to the (Z)b-B group:

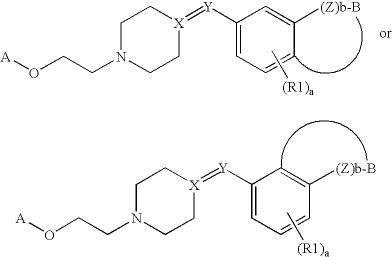

When Z is oxygen, CH₂ or CH, and B forms the rest of an aryl group fused to the phenyl ring, preferably the aryl group is a 5 or 6 membered group containing 1, 2 or 3 heteroatoms selected from nitrogen, sulphur and oxygen, optionally substituted by 1 to 3 groups selected from halogen, C₁₋₆alkyl, CF₃, cyano, hydroxy, C₁₋₆alkanoyl and C₁₋₆alkoxy.

When Z is oxygen, CH₂ or CH, and B forms the rest of a C₃₋₇heterocyclic group fused to the phenyl ring, preferably the heterocyclic group is a saturated 5 or 6 membered group containing 1, 2 or 3 heteroatoms selected from nitrogen, sulphur and oxygen, the heterocyclic group being optionally substituted by 1 to 3 groups selected from halogen, oxo, C₁₋₆alkyl, cyano, CF₃, C₁₋₆alkoxy and C₁₋₆alkanoyl.

Examples of groups formed when Z is oxygen, CH or CH₂, and together with B, forms an aryl or a 3 to 7 membered heterocyclic group which is fused to the phenyl ring in formula (I) include: indene, naphthalene, indole, isoindole, benzodioxoline, 3H-indole, indoline, benzofuran, benzothiophene, 1H-indazole, benzimidazole, benzthiazole, quinoline, isoquinoline, quinazoline, quinoxaline, chroman, isochroman, benzoxazine, and benzooxazoline. These groups may be substituted as described above.

Preferred compounds of this invention are compounds of Examples 1-79 (as described below) and pharmaceutically acceptable salts thereof.

The compounds of formula (I) can form acid addition salts thereof. It will be appreciated that for use in medicine the salts of the compounds of formula (I) should be pharmaceutically acceptable. Suitable pharmaceutically acceptable salts will be apparent to those skilled in the art and include those described in J. Pharm. Sci., 1977, 66, 1-19, such as acid addition salts formed with inorganic acids e.g. hydrochloric, hydrobromic, sulfuric, nitric or phosphoric acid; and organic acids e.g. succinic, maleic, acetic, fumaric, citric, tartaric, benzoic, p-toluenesulfonic, methanesulfonic or naphthalenesulfonic acid. Certain of the compounds of formula (I) may form acid addition salts with one or more equivalents of the acid. The present invention includes within its scope all possible stoichiometric and non-stoichiometric forms.

The compounds of formula (I) may be prepared in crystalline or non-crystalline form, and, if crystalline, may optionally be hydrated or solvated. This invention includes within its scope stoichiometric hydrates or solvates as well as compounds containing variable amounts of water and/or solvent.

Certain compounds of formula (I) are capable of existing in stereoisomeric forms (e.g. geometric or (“cis-trans”) isomers, diastereomers and enantiomers) and the invention extends to each of these stereoisomeric forms and to mixtures thereof including racemates. The different stereoisomeric forms may be separated one from the other by the usual methods, or any given isomer may be obtained by stereospecific or asymmetric synthesis. The invention also extends to any tautomeric forms and mixtures thereof. The present invention includes within its scope all such isomers, including mixtures.

In a further aspect, this invention provides a process for the preparation of a compound of formula (I) or a pharmaceutically acceptable salt thereof, which process comprises:
(a) the coupling of a compound of formula (II): 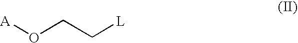
   wherein A is as defined for formula (I) and L is a leaving group,
with a compound of formula (III): 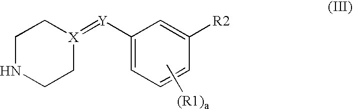
   wherein a, X, Y, R1, R2 and are as defined for formula (I); or
(b) for a compound wherein X is nitrogen, the coupling of a compound of formula (IV): 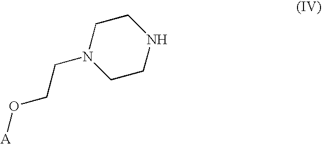
   wherein A has the same meanings as for formula (I), and a compound of formula (V): 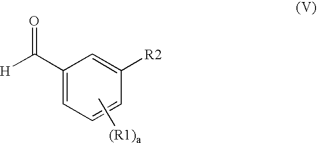
   wherein a, R1, and R2 have the same meanings as for formula (I),
and thereafter optionally for either process (a) or (b):
removing any protecting groups and/or
converting a compound of formula (I) into another compound of formula (I) and/or
forming a pharmaceutically acceptable salt.

For process (a), the reaction of compounds of formulae (II) and (III) is preferably carried out in a suitable solvent such as isopropyl alcohol or N,N-dimethylformamide, in the presence of an appropriate base such as N,N-diisopropylethylamine or potassium carbonate. A suitable leaving group L is bromine.

For process (b), the reaction of compounds of formulae (IV) and (V) is preferably carried out in an aprotic solvent such as 1,2-dichloroethane, in the presence of an appropriate reducing agent such as sodium triacetoxyborohydride.

Compounds of formula (I) can be converted into further compounds of formula (I) using standard techniques. For example, and by way of illustration rather than limitation, for compounds of formula (I) wherein ═ is a double bond can be converted to compounds of formula (I) in which ═ is a single bond by palladium catalysed hydrogenation in a suitable solvent such as ethanol. Other possible conversion reactions include acylation with an appropriate acylating agent such as acetyl chloride, alkylation using an appropriate alkylating reagent such as methyl iodide, and sulfonylation using a sulfonylating agent such as methanesulfonic anhydride.

Compounds of formulae (II), (III), (IV) and (V) are commercially available, may be prepared according to procedures described herein, by known literature methods, or by analogous procedures thereto.

For example, for compounds of the present invention wherein X is carbon, Y is CH and ═ is a double bond, compounds of formula (III) wherein X is carbon may be prepared by reacting a compound of formula (VI): 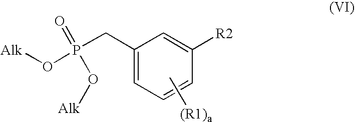
wherein “Alk” refers to an alkyl group, with a compound of formula (VII): 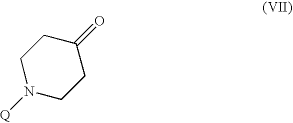
wherein Q is a protecting group such as t-butyloxycarbonyl, in the presence of a base such as sodium hydride, in a solvent such as tetrahydrofuran or N,N-dimethylformamide. The protecting group Q may be removed thereafter by any suitable means.

Compounds of formula (VI) may be prepared by treating a compound of formula (VIII): 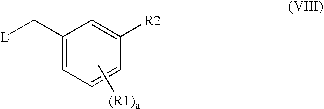
wherein L is a leaving group such as bromine, with a trialkyl phosphite such as triethyl phosphite or trimethyl phosphite, in the absence of solvent or in the presence of a solvent such as toluene.

It will be appreciated by those skilled in the art that it may be necessary to protect certain reactive substituents during some of the above procedures. Standard protection and deprotection techniques, such as those described in Greene T. W. Protective groups in organic synthesis, New York, Wiley (1981), can be used. For example, primary amines can be protected as phthalimide, benzyl, t-butyloxycarbonyl, benzyloxycarbonyl or trityl derivatives. Carboxylic acid groups can be protected as esters. Aldehyde or ketone groups can be protected as acetals, ketals, thioacetals or thioketals. Deprotection of such groups is achieved using conventional procedures well known in the art. For example, protecting groups such as t-butyloxycarbonyl may be removed using an acid such as hydrochloric or trifluroroacetic acid in a suitable solvent such as dichloromethane, diethylether, isopropanol or mixtures thereof.

Pharmaceutically acceptable salts may be prepared conventionally by reaction with the appropriate acid or acid derivative.

The affinities of the compounds of this invention for 5-HT_{1A}, 5-HT_{1B} and 5-HT_{1D} receptors can be determined by the radioligand binding assay as described in WO 99/07700. All compounds tested according to the radioligand binding assay described above were found to have pKi values >6.0 at 5-HT_{1A}, 5-HT_{1B} and 5-HT_{1D} receptors, with many showing a considerably higher affinity (having pKi values in the range 8.0-10.0).

The intrinsic activity of the compounds of this invention can be determined according to the [³⁵S]GTPγS functional assay which is also described in WO 99/07700. It has been found, using the [³⁵S]GTPγS functional assay, that certain compounds of formula (I) appear to be antagonists at 5-HT₁ type receptors whilst others appear to be inverse agonists, agonists or partial agonists.

Compounds of formula (I) and their pharmaceutically acceptable salts are of use in the treatment of certain CNS disorders such as depression (both bipolar and unipolar), single or recurrent major depressive episodes with or without psychotic features, catatonic features, melancholic features, atypical features or postpartum onset, seasonal affective disorder and dysthymia, anxiety disorders, including generalised anxiety, schizophrenia, panic disorder, agoraphobia, social phobia, obsessive compulsive disorder and post-traumatic stress disorder; pain (particularly neuropathic pain); memory disorders, including dementia, amnesic disorders and age-associated memory impairment; disorders of eating behaviours, including anorexia nervosa and bulimia nervosa, sexual dysfunction, sleep disorders (including disturbances of circadian rhythm, dyssomnia, insomnia, sleep apnea and narcolepsy), withdrawal from abuse of drugs such as of cocaine, ethanol, nicotine, benzodiazepines, alcohol, caffeine, phencyclidine (phencyclidine-like compounds), opiates (e.g. cannabis, heroin, morphine), sedative ipnotic, amphetamine or amphetamine-related drugs (e.g. dextroamphetamine, methylamphetamine) or a combination thereof, motor disorders such as Parkinson's disease, dementia in Parkinson's disease, neuroleptic-induced Parkinsonism and tardive dyskinesias, as well as other psychiatric disorders. Depressive disorders which may be treated or prevented by the compounds of formula (I) and their pharmaceutically acceptable salts may also result from a general medical condition including, but not limited to, myocardial infarction, diabetes, miscarriage or abortion, etc. Compounds of formula (I) may also have utility in the treatment of certain gastrointestinal disorders such as irritable bowel syndrome.

It is to be understood that “treatment” as used herein includes prophylaxis as well as alleviation of established symptoms.

Thus the invention also provides a compound of formula (I) or a pharmaceutically acceptable salt thereof, for use in therapy, particularly for a CNS disorder such as depression or anxiety.

In a further aspect, the present invention provides a method of treatment of the above disorders, particularly a CNS disorder such as depression or anxiety, in mammals including humans, which comprises administering to the sufferer a therapeutically safe and effective amount of a compound of formula (I) or a pharmaceutically acceptable salt thereof.

In another aspect, the invention provides for the use of a compound of formula (I) or a pharmaceutically acceptable salt thereof in the manufacture of a medicament for use in the treatment of the above disorders, particularly a CNS disorder such as depression or anxiety.

In another aspect, the present invention provides the use of a compound of formula (Ia) or a pharmaceutically acceptable salt thereof: 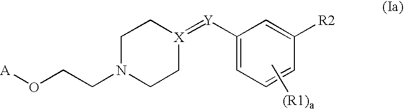
wherein
A is optionally substituted phenyl, indolyl, quinolinyl, quinazolinyl, indazolyl or isoquinolinyl;
X is carbon, Y is CH and ═ is a double bond; or X is CH, Y is CH₂ or oxygen and ═ is a single bond; or X is nitrogen, Y is CH₂ and ═ is a single bond;
R1 is halogen, cyano, nitro, C₁₋₆alkyl, haloC₁₋₆alkyl, C₁₋₆alkoxy, C₃₋₇heterocyclylC₁₋₆alkyl, C₃₋₇heterocyclyl C₁₋₆alkoxy;
d is 0, 1, 2, 3 or 4;
R2 is either:
   halogen, —CN, C₃₋₇cycloalkyl, aryl or a C-linked 3-7 membered heterocyclic group; or
   a group -(Z)b-B wherein:
      (i) Z is oxygen, CH₂, C═O, SO₂ or C═N—OR3 wherein R3 is hydrogen or C₁₋₆alkyl; b is 1, 2, or 3; and B is hydrogen, C₁₋₆alkyl, C₃₋₇cycloalkyl, C₁₋₆alkoxy or NR4R5 wherein R4 and R5 are independently hydrogen, C₁₋₆alkyl, C₃₋₇cycloalkyl, C₁₋₆alkanoyl, fluoroC₁₋₆alkanoyl, C₁₋₆alkylsulfonyl, fluoroC₁₋₆alkylsulfonyl, carbamoyl or C₁₋₆alkylcarbamoyl, or R4 and R5, together with the nitrogen atom to which they are attached, form part of an optionally substituted 3 to 7 membered heterocyclic group; or
      (ii) Z is oxygen, CH or CH₂, b is 1, and B forms the rest of an aryl or a C₃₋₇heterocyclic group fused to the phenyl ring;
in the manufacture of a medicament for use in the treatment of depression and/or anxiety.

All preferred features of formula (I) apply to formula (Ia) mutatis mutandis. Compounds of formula (la) may be prepared in the same manner as for compounds of formula (I).

In a further aspect, the present invention provides a method of treatment of depression or anxiety, in mammals including humans, which comprises administering to the sufferer a therapeutically safe and effective amount of a compound of formula (Ia) or a pharmaceutically acceptable salt thereof.

Compounds of the invention may be administered in combination with other active substances such as 5HT3 antagonists, serotonin agonists, NK-1 antagonists, selective serotonin reuptake inhibitors (SSRI), noradrenaline re-uptake inhibitors (SNRI), tricyclic antidepressants and/or dopaminergic antidepressants.

Suitable 5HT3 antagonists which may be used in combination of the compounds of the inventions include for example ondansetron, granisetron, metoclopramide.

Suitable serotonin agonists which may be used in combination with the compounds of the invention include sumatriptan, rauwolscine, yohimbine, metoclopramide.

Suitable SSRIs which may be used in combination with the compounds of the invention include fluoxetine, citalopram, femoxetine, fluvoxamine, paroxetine, indalpine, sertraline, zimeldine.

Suitable SNRIs which may be used in combination with the compounds of the invention include venlafaxine and reboxetine.

Suitable tricyclic antidepressants which may be used in combination with a compound of the invention include imipramine, amitriptiline, chlomipramine and nortriptiline.

Suitable dopaminergic antidepressants which may be used in combination with a compound of the invention include bupropion and amineptine.

It will be appreciated that the compounds of the combination or composition may be administered simultaneously (either in the same or different pharmaceutical formulations), separately or sequentially.

In order to use the compounds of the present invention in therapy, they will normally be formulated into a pharmaceutical composition in accordance with standard pharmaceutical practice. The present invention also provides a pharmaceutical composition, which comprises a compound of the present invention or a pharmaceutically acceptable salt thereof, and a pharmaceutically acceptable carrier or excipient.

In a further aspect, the present invention provides a process for preparing a pharmaceutical composition, the process comprising mixing a compound of the present invention or a pharmaceutically acceptable salt thereof and a pharmaceutically acceptable carrier or excipient.

A pharmaceutical composition of the invention, which may be prepared by admixture, suitably at ambient temperature and atmospheric pressure, is usually adapted for oral, parenteral or rectal administration and, as such, may be in the form of tablets, capsules, oral liquid preparations, powders, granules, lozenges, reconstitutable powders, injectable or infusible solutions or suspensions or suppositories. Orally administrable compositions are generally preferred.

Tablets and capsules for oral administration may be in unit dose form, and may contain conventional excipients, such as binding agents (e.g. pregelatinised maize starch, polyvinylpyrrolidone or hydroxypropyl methylcellulose);, fillers (e.g. lactose, microcrystalline cellulose or calcium hydrogen phosphate);, tabletting lubricants lubricants (e.g. magnesium stearate, talc or silica);, disintegrants (e.g. potato starch or sodium starch glycollate); and acceptable wetting agents (e.g. sodium lauryl sulphate). The tablets may be coated according to methods well known in normal pharmaceutical practice.

Oral liquid preparations may be in the form of, for example, aqueous or oily suspension, solutions, emulsions, syrups or elixirs, or may be in the form of a dry product for reconstitution with water or other suitable vehicle before use. Such liquid preparations may contain conventional additives such as suspending agents (e.g. sorbitol syrup, cellulose derivatives or hydrogenated edible fats), emulsifying agents (e.g. lecithin or acacia), non-aqueous vehicles (which may include edible oils e.g. almond oil, oily esters, ethyl alcohol or fractionated vegetable oils), preservatives (e.g. methyl or propyl-p-hydroxybenzoates or sorbic acid), and, if desired, conventional flavourings or colorants, buffer salts and sweetening agents as appropriate. Preparations for oral administration may be suitably formulated to give controlled release of the active compound.

For parenteral administration, fluid unit dosage forms are prepared utilising a compound of the invention or pharmaceutically acceptable salt thereof and a sterile vehicle. Formulations for injection may be presented in unit dosage form e.g. in ampoules or in multi-dose, utilising a compound of the invention or pharmaceutically acceptable salt thereof and a sterile vehicle, optionally with an added preservative. The compositions may take such forms as suspensions, solutions or emulsions in oily or aqueous vehicles, and may contain formulatory agents such as suspending, stabilising and/or dispersing agents. Alternatively, the active ingredient may be in powder form for constitution with a suitable vehicle, e.g. sterile pyrogen-free water, before use. The compound, depending on the vehicle and concentration used, can be either suspended or dissolved in the vehicle. In preparing solutions, the compound can be dissolved for injection and filter sterilised before filling into a suitable vial or ampoule and sealing. Advantageously, adjuvants such as a local anaesthetic, preservatives and buffering agents are dissolved in the vehicle. To enhance the stability, the composition can be frozen after filling into the vial and the water removed under vacuum. Parenteral suspensions are prepared in substantially the same manner, except that the compound is suspended in the vehicle instead of being dissolved, and sterilisation cannot be accomplished by filtration. The compound can be sterilised by exposure to ethylene oxide before suspension in a sterile vehicle. Advantageously, a surfactant or wetting agent is included in the composition to facilitate uniform distribution of the compound.

Lotions may be formulated with an aqueous or oily base and will in general also contain one or more emulsifying agents, stabilising agents, dispersing agents, suspending agents, thickening agents, or colouring agents. Drops may be formulated with an aqueous or non-aqueous base also comprising one or more dispersing agents, stabilising agents, solubilising agents or suspending agents. They may also contain a preservative.

The compounds of the invention may also be formulated in rectal compositions such as suppositories or retention enemas, e.g. containing conventional suppository bases such as cocoa butter or other glycerides.

The compounds of the invention may also be formulated as depot preparations. Such long acting formulations may be administered by implantation (for example subcutaneously or intramuscularly) or by intramuscular injection. Thus, for example, the compounds of the invention may be formulated with suitable polymeric or hydrophobic materials (for example as an emulsion in an acceptable oil) or ion exchange resins, or as sparingly soluble derivatives, for example, as a sparingly soluble salt.

For intranasal administration, the compounds of the invention may be formulated as solutions for administration via a suitable metered or unitary dose device or alternatively as a powder mix with a suitable carrier for administration using a suitable delivery device. Thus compounds of formula (la) may be formulated for oral, buccal, parenteral, topical (including ophthalmic and nasal), depot or rectal administration or in a form suitable for administration by inhalation or insufflation (either through the mouth or nose).

The compounds of the invention may be formulated for topical administration in the form of ointments, creams, gels, lotions, pessaries, aerosols or drops (e.g. eye, ear or nose drops). Ointments and creams may, for example, be formulated with an aqueous or oily base with the addition of suitable thickening and/or gelling agents. Ointments for administration to the eye may be manufactured in a sterile manner using sterilised components.

The composition may contain from 0.1% to 99% by weight, preferably from 10 to 60% by weight, of the active material, depending on the method of administration. The dose of the compound used in the treatment of the aforementioned disorders will vary in the usual way with the seriousness of the disorders, the weight of the sufferer, and other similar factors. However, as a general guide suitable unit doses may be 0.05 to 1000 mg, more suitably 1.0 to 200 mg, and such unit doses may be administered more than once a day, for example two or three times a day. Such therapy may extend for a number of weeks or months.

All publications, including but not limited to patents and patent applications, cited in this specification are herein incorporated by reference as if each individual publication were specifically and individually indicated to be incorporated by reference herein as though fully set forth.

The following Descriptions and Examples illustrate the preparation of compounds of the invention.

### Description 1

### tert-Butyl 4-(3-pyridin-4-ylbenzylidene)piperidine-1-carboxylate (D1)

A stirred solution of tert-butyl 4-(3-bromobenzylidene)piperidine-1-carboxylate (0.35 g, 1.0 mmol), 4-pyridineboronic acid (0.14 g, 1.1 mmol), sodium carbonate (0.32 g, 3.0 mmol) and tetrakis(triphenylphosphine)palladium (0) (0.05 g, 0.04 mmol) in 50% aq. ethylene glycol dimethyl ether (10 mL) was heated to reflux for 18 h. The cooled mixture was concentrated to near dryness and diluted with water (10 mL) and extracted with dichloromethane. The extracts were dried and concentrated to dryness in vacuo, the residue was subjected to chromatography on silica eluting with 1% methanol in dichloromethane, followed by a second column using 50% diethyl ether/40-60 petroleum ether as the eluant. This afforded the title compound as a colourless gum (0.19 g, 54%) Mass spectrum (API⁺): Found 351 (MH⁺). C₂₂H₂₆N₂O₂ requires 350.

¹H NMR (CDCl₃) δ: 1.48 (9H, s), 2.37 (2H, t), 2.49 (2H, t), 3.42 (2H, t), 3.53 (2H, t), 6.42 (1H, s), 7.26 (1H, s), 7.42-7.50 (5H, m), 8.65 (2H, d,).

### Description 2

### tert-Butyl 4-(3-pyridinium-4-ylbenzylidene)piperidine-1-carboxylate iodide (D2)

A solution of tert-butyl 4-(3-pyridin-4-ylbenzylidene)piperidine-1-carboxylate (0.19 g, 0.54 mmol) in dichloromethane (10 mL) was treated with iodomethane (0.08 g, 0.06 mmol). After 24 h at room temp a further 0.24 g (0.18 mmol) of iodomethane was added. After 72 h the solvent and excess reagent was removed by evaporation in vacuo to afford the crude title compound as a yellow solid (0.22 g, 85%).

### Description 3

### tert-Butyl 4-[3-(1-methylpiperidin-4-yl)benzyl]piperidine-1-carboxylate (D3)

A mixture of the crude tert-butyl 4-(3-pyridinium-4-ylbenzylidene)piperidine-1-carboxylate iodide (0.21 g, 0.43 mmol) and platinum oxide (0.03 g, 0.13 mmol) in ethanol (10 mL) was shaken under an atmosphere of hydrogen at 50 psi for 24 h. The mixture was filtered and concentrated to dryness in vacuo to afford the crude title compound (0.14 g, 88%).

Mass spectrum (API⁺): Found 373 (MH⁺). C₂₃H36N₂O₂ requires 372.

### Description 4

### 4-[3-(1-Methylpiperidin-4-yl)benzyl]piperidine hydrochloride (D4)

A mixture of the crude tert-butyl 4-[3-(1-methylpiperidin4-yl)benzyl]piperidine-1-carboxylate (0.14 g, 0.38 mmol) and 1.0 M HCl/diethyl ether (10 mL) in methanol (10 mL) was stirred at room temp for 18 h. Concentration of the mixture in vacuo afforded the title compound as a colourless gum (0.16 g, >100%).

¹H NMR (CDCl₃) δ: 1.21-1.36 (2H, m), 1.61 (2H, br s), 1.85 (3H, br d), 2.04 (2H, br d), 2.42 (2H, br s,), 2.60 (2H, s), 2.72 (4H,br s), 2.86 (4H,m), 3.40 (1H, s), 3.58 (2H, br s), 7.02-7.12 (2H, m,), 7.23-7.33 (2H, m).

### Description 5

### 2-Chloro-5-(pyridin-4-ylmethyl)benzenesulfonyl chloride (D5)

Chlorosulfonic acid (8 mL) was slowly added to 4-(4-chlorophenylmethyl)pyridine (5.0 g, 0.0245 mol) in a 100 mL round bottom flask under argon, with stirring. Sodium chloride (1.4 g, 0.0245 mol) was added and the mixture stirred at 70° C. for 24 h. The mixture was cooled and poured onto ice forming a precipitate which was collected by filtration and dried to give the title compound (5.12 g, 69%) as a solid.

Mass spectrum (API⁺): Found 302 (MH⁺). C₁₂H₉³⁵Cl₂NO₂S requires 301.

¹H NMR (d⁶DMSO) δ: 4.31 (2H, s), 7.30 (1H, dd, J=8, 2 Hz), 7.37 (1H, d, J=8 Hz), 7.82 (1H, d, J=2 Hz), 7.95 (2H, d, J=7 Hz), 8.83 (2H, d, J=7 Hz).

### Description 6

### 2-Chloro-5-(pyridin-4-ylmethyl)benzenesulfonamide (D6)

A solution of 2-chloro-5-(pyridin-4-ylmethyl)benzenesulfonyl chloride (1.95 g, 0.0065 mol) in concentrated ammonium hydroxide solution (40 mL) was stirred at 20° C. for 2 h, and then cooled to 5° C. in ice. The mixture was filtered and the filtrate evaporated in vacuo to give the title compound (1.84 g, 100%) as a solid.

Mass spectrum (API⁺): Found 283 (MH⁺). C₁₂H₁₁³⁵ClN₂O₂S requires 282.

### Description 7

### 2-Chloro-5-(piperidin-4-ylmethyl)benzenesulfonamide hydrochloride (D7)

A mixture of 2-chloro-5-(pyridin-4-ylmethyl)benzenesulfonamide (1.0 g, 0.0035 mol), 1 M HCl in ether (5 mL) and methanol (40 mL) was stirred under an atmosphere of hydrogen (1 atm) over platinum (IV) oxide (0.1 g) for 36 h. The mixture was filtered through celite and the filtrated evaporated in vacuo to give the title compound (1.14 g, 100%) as a solid.

Mass spectrum (API⁺): Found 289 (MH⁺). C₁₂H₁₇³⁵ClN₂O₂S requires 288.

### Description 8

### 3-(Piperidin-4-ylmethyl)benzenesulfonamide hydrochloride (D8)

A solution of 2-chloro-5-(piperidin-4-ylmethyl)benzenesulfonamide hydrochloride (1.0 g, 0.0032 mol) in methanol (15 mL) was hydrogenated over 10% palladium on charcoal (0.01 g), at 50 psi and 50° C. for 48 h. The mixture was cooled and filtered through celite and the filtrate evaporated in vacuo to give the title compound (0.93 g, 100%) as a solid.

Mass spectrum (API⁺): Found 255 (MH⁺). C₁₂H₁₈N₂O₃S requires 254.

### Description 9

### 2-(5-Quinolinyloxy)ethyl bromide (D9)

A mixture of 5-hydroxyquinoline (0.3 g, 2.1 mmol), 1,2-dibromoethane (3.9 g, 21 mmol) and potassium carbonate (1.5 g, 11 mmol) in methyl ethyl ketone (15 mL) was allowed to stir at 85° C. for 24 h. The mixture was evaporated in vacuo and the residue was partitioned between ether (200 mL) and water (200 mL). The organic layer was dried over sodium sulfate and evaporated in vacuo to give the title compound (0.53 g).

¹H NMR (CDCl₃) δ: 3.80 (2H, m), 4.49 (2H, m), 6.86 (1H, d, J=8 Hz), 7.41 (1H, dd, J=8, 4 Hz), 7.61 (1H, t, J=8 Hz), 7.73 (1H, d, J=8 Hz), 8.64 (1H, d, J=8 Hz), 8.91 (1H, m).

### Description 10

### 5-Hydroxy-2-methylquinoline (D10)

A mixture of 2-methyl-5,6,7,8-tetrahydroquinolin-5-one [E. Reimann, J. Freisinger, Arch. Pharm. (Weinheim), 318, 871 (1985)] (0.57 g, 3.5 mmol) and 48% aqueous HBr (3.5 mL) was warmed to 60° C. and treated dropwise with bromine (0.19 mL, 0.59 g, 3.6 mmol), with vigorous stirring. The resulting mixture was stirred at 60° C. for 1 h, then evaporated in vacuo. The residue was treated with isopropanol with stirring, then the mixture was evaporated in vacuo to give a waxy solid, which was triturated with 1:1 isopropanol—ether to give a beige powder (0.9 g). A mixture of this material, lithium carbonate (0.48 g, 6.7 mmol), lithium bromide (0.28 g, 3.2 mmol) and N,N-dimethylformamide (10 mL) was heated at 150° C. under argon with stirring for 2 h. The mixture was cooled then evaporated in vacuo. Chromatography of the residue on silica with 0-100% ethyl acetate-hexane gradient elution gave the title compound (0.28 g, 49%) as a solid.

Mass spectrum (API⁺): Found 160 (MH⁺). C₁₀H₉NO requires 159.

### Description 11

### 5-(2-Bromoethoxy)-2-methylquinoline (D11)

The title compound was prepared from 5hydroxy-2-methylquinoline and 1,2-dibromoethane using a similar procedure to Description 9, in 91% yield.

Mass spectrum (API⁺): Found 266 (MH⁺). C₁₂H₁₂⁷⁹BrNO requires 265.

### Description 12

### Diethyl (3-cyanobenzyl)phosphonate (D12)

A mixture of 3-cyanobenzyl bromide (7.8 g, 0.04 mol), triethyl phosphite (6.6 mL, 0.04 mol) in toluene (200 mL) was stirred at reflux for 24 h. The reaction mixture was cooled and evaporated in vacuo. Chromatography of the residues on SiO₂ eluting from 0-100% ethyl acetate in petroleum ether (60-80° C.) gave the title compound (7.2 g, 71%) as a yellow liquid.

Mass spectrum (API⁺): Found 254 (MH⁺). C₁₂H₁₆NO₃P requires 253.

### Description 13

### Diethyl (3-methoxycarbonylbenzyl)phosphonate (D13)

The title compound was prepared in a similar manner to Description 12.

Mass spectrum (API⁺): Found 287 (MH⁺). C₁₃H₁₉PO₅ requires 286.

### Description 14

### Diethyl (3-methoxybenzyl)phosphonate (D14)

The title compound was prepared in a similar manner to Description 12.

Mass spectrum (API⁺): Found 259 (MH⁺). C₁₂H₁₉PO₄ requires 258.

### Description 15

### Diethyl (3-bromobenzyl)phosphonate (D15)

The title compound was prepared in a similar manner to description D12.

Mass spectrum (API⁺): Found 307 (MH⁺). C₁₁H₁₆⁷⁹BrPO₃ requires 306.

### Description 16

### tert-Butyl 4-(3-cyanobenzylidene)piperidine-1-carboxylate (D16)

A mixture of diethyl (3-cyanobenzyl)phosphonate (3 g, 0.012 mol) and tert-butyl 4-oxo-piperidine-1-carboxylate (2.1 g, 0.012 mol) in dry tetrahydrofuran (50 mL) was treated with a 60% suspension of sodium hydride in oil (0.43 g, 0.012 mmol). The resulting mixture was stirred at room temperature for 4 h, then partitioned between dichloromethane (500 mL) and water (500 mL). The organic extract was dried (Na₂SO₄) and evaporated in vacuo to give the title compound (4.2 g, 100%) as a solid.

¹H NMR (CDCl₃) δ: 1.48 (9H, s), 2.33-2.44 (4H, m), 3.39-3.44 (2H, m), 3.50-3.54 (2H, m), 6.32 (1H, s), 7.40-7.50 (4H, m).

### Description 17

### tert-Butyl 4-(3-methoxycarbonylbenzylidene)piperidine-1-carboxylate (D17)

The title compound was prepared from diethyl (3-methoxycarbonylbenzyl)phosphonate in a similar manner to Description 16.

¹H NMR (CDCl₃) δ: 1.48 (9H, s), 2.35 (2H, m), 2.44 (2H, m), 3.41 (2H, m), 3.52 (2H, m), 3.92 (3H, s), 6.34 (1H, s), 7.38 (2H, m), 7.88 (2H, m).

### Description 18

### tert-Butyl 4-(3-methoxybenzylidene)piperidine-1-carboxylate (D18)

The title compound was prepared from diethyl (3-methoxybenzyl)phosphonate in a similar manner to Description 16.

¹H NMR (CDCl₃) δ: 1.49 (9H, s), 2.33 (2H, m), 2.44 (2H, m), 3.42 (2H, m), 3.51 (2H, m), 3.80 (3H, s), 6.33 (1H, s), 6.74 (1H, m), 6.87 (2H, m), 7.22 (1H, m).

### Description 19

### tert-Butyl 4-(3-bromobenzylidene)piperidine-1-carboxylate (D19)

The title compound was prepared from diethyl (3-bromobenzyl)phosphonate in a similar manner to Description 16.

¹H NMR (CDCl₃) δ: 1.48 (9H, s), 2.33 (2H, m), 2.43 (2H, m), 3.41 (2H, m), 3.50 (2H, m), 6.29 (1H, s), 7.11 (1H, m), 7.18 (1H, t, J=8 Hz), 7.34 (2H, m).

### Description 20

### 4-(3-Cyanobenzylidene)piperidine hydrochloride (D20)

A mixture of tert-butyl 4-(3-cyanobenzylidene)piperidine-1-carboxylate (4.2 g, 0.014 mol), methanol (5 mL) and 1M HCl in diethyl ether (100 mL) was stirred at 40° C. for 10 h. The reaction mixture was evaporated in vacuo and the residue triturated in diethyl ether (3×100 mL) to give the title compound (1.4 g, 50%) as a solid.

Mass spectrum (API⁺): Found 199 (MH⁺). C₁₃H₁₄N₂ requires 198.

### Description 21

### 4-(3-Methoxycarbonylbenzylidene)piperidine hydrochloride (D21)

The title compound was prepared from tert-butyl 4-(3-methoxycarbonylbenzylidene)piperidine-1-carboxylate in a similar manner to Description 20.

¹H NMR (CD₃OD) δ: 2.67 (2H, m), 2.72 (2H, m), 3.20 (3H, m), 3.30 (2H, m), 3.91 (3H, s), 6.60 (1H, s), 7.48 (2H, m), 7.87 (1H, s), 7.91 (1H, m).

### Description 22

### 4-(3-Methoxybenzylidene)piperidine hydrochloride (D22)

The title compound was prepared from tert-butyl 4-(3-methoxybenzylidene)piperidine-1-carboxylate in a similar manner to Description 20.

Mass spectrum (API⁺): Found 204 (MH⁺). C₁₃H₁₇NO requires 203.

### Description 23

### 4-(3-Bromobenzylidene)piperidine hydrochloride (D23)

The title compound was prepared from tert-butyl 4-(3-bromobenzylidene)piperidine-1-carboxylate in a similar manner to Description 20.

Mass spectrum (API⁺): Found 252 (MH⁺). C₁₂H₁₄⁷⁹BrN requires 251.

### Description 24

### Methyl 3-(1-(2-(2-methylquinolin-5-yloxy)ethyl)piperidin-4-ylidenemethyl)benzoate (D24)

The title compound was prepared from 4-(3-methoxycarbonylbenzylidene)piperidine hydrochloride and 5-(2-bromoethoxy)-2-methylquinoline in a similar manner to Example 5.

Mass spectrum (API⁺): Found 417 (MH⁺). C₂₆H₂₈N₂O₃ requires 416.

### Description 25

### tert-Butyl 4-(3-cyanobenzyl)piperidine-1-carboxylate (D25)

A mixture of tert-butyl 4-(3-cyanobenzylidene)piperidine-1-carboxylate (0.5 g, 1.7 mmol) and 10% palladium on charcoal (paste) (0.05 g) in methanol (40 mL) was stirred at 20° C., under an atmosphere of hydrogen (1 atm) for 24 h. The reaction mixture was filtered through celite and the filtrate evaporated in vacuo to give the title compound (0.5 g, 100%) as a solid.

### Description 26

### 3-(Piperidin-4-ylmethyl)benzamide hydrochloride (D26)

A solution of tert-butyl 4-(3-cyanobenzyl)piperidine-1-carboxylate (0.5 g, 0.0017 mol) in dichloromethane (40 mL) was treated with trifluoromethanesulfonic anhydride (0.47 g, 0.0017 mol) and stirred at 20° C. for 2 h. Saturated sodium bicarbonate solution (10 mL) was added and stirring continued for 4 h. The organic layer was dried (Na₂SO₄) and evaporated in vacuo. The residue was dissolved in 1 N HCl in ether (20 mL) and stirred at 20° C. for 24 h. The mixture was evaporated in vacuo to give the title compound (0.21 g, 50%) as a solid.

Mass spectrum (API⁺): Found 219 (MH⁺). C₁₃H₁₈N₂O requires 218.

### Description 27

### tert-Butyl 4-[2-(2-methylquinolin-5-yloxy)ethyl]piperazine-1-carboxylate (D27)

tert-Butyl piperazine-1-carboxylate (1.4 g, 7.52 mmol) was added to a mixture of 5-(2-bromoethoxy)-2-methylquinoline (2 g, 7.52 mmol) and potassium carbonate (4.16 g, 30.1 mmol) in N,N-dimethylformamide (20 mL). The reactants were heated at 70° C. for 16 h under an atmosphere of argon. The reaction mixture was poured into water (200 mL) and extracted into ethyl acetate (3×200 mL). The organic layers were combined, dried (Na₂SO₄) and concentrated in vacuo. The residue was purified by column chromatography, eluting with 30% ethyl acetate in hexane affording the title compound as a tan solid (1.04 g, 37%).

Mass spectrum (API⁺): Found 372.3 (MH⁺). C₂₁H29N₃O₃ requires 371.

¹H NMR (CDCl₃) δ: 1.46 (9H, s), 2.59 (4H, t), 2.73 (3H, s), 2.96 (2H, t), 3.46 (4H, t), 4.29 (2H, t), 6.80 (1H, dd), 7.26 (1H, d), 7.58 (2H, m), 8.43 (1H, d).

### Description 28

### 2-Methyl-5-(2-piperazin-1-ylethoxy)quinoline (D28)

tert-Butyl 4-[2-(2-methylquinolin-5-yloxy)ethyl]piperazine-1-carboxylate (1.04 g, 2.8 mmol) was dissolved in ethanol (60 mL) and treated with 1 M hydrochloric acid in diethyl ether (16 mL, 16 mmol) and stirred at 40° C. for 17 h. The reaction mixture was filtered and the white solid was collected and dried in vacuo. The hydrochloride salt precipitate was dissolved in water (25 mL) and potassium carbonate was added until the pH reached 10. The aqueous layer was extracted with 5% methanol in dichloromethane (4×100 mL) then 10% methanol in dichloromethane (4×100 mL). The organic layers were combined, dried (Na₂SO₄) and concentrated in vacuo, affording the title compound as a brown oil (0.69 g, 91%).

Mass spectrum (API⁺): Found 272 (MH⁺). C₁₆H₂₁N₃O requires 271.

¹H NMR (CDCl3) δ: 2.62 (4H, m), 2.73 (3H, s), 2.92 (6H, m), 3.47 (1H, s), 4.29 (2H, d), 6.80 (1H, dd), 7.50 (1H, d), 7.58 (2H, m), 8.45 (1H, d).

### Description 29

### Diethyl 3-fluoro-4-methoxybenzylphosphonate (D29)

A mixture of 3-fluoro-4-methoxybenzyl chloride [Cervena, Irena; Holubek, Jiri; Svatek, Emil; Valchar, Martin; Protiva, Miroslav; Collect.Czech.Chem.Commun.; 52; 10; 1987; 2564-2571.] (6 g, 35 mmol) and triethylphosphite (23 g, 140 mmol) was stirred under reflux for 16 hours. Removal of the excess triethylphosphite in vacuo gave the title compound (11.2 g, 100%) as an amber oil.

Mass spectrum (API⁺): Found 277 (MH⁺). C₁₂H₁₈FO₄P requires 276.

¹H NMR (CDCl₃) δ: 1.26 (6H, m), 3.06 (2H, d, J=21 Hz), 3.87 (3H, s), 3.90-4.10 (4H, m), 6.90 (1H, t, J=8 Hz), 6.95-7.05 (2H, m).

### Description 30

### tert-Butyl 4-(3-fluoro-4-methoxybenzylidene)piperidine-1-carboxylate (D30)

A 1M solution of potassium tert-butoxide in THF (24 mL, 24 mmol) was added dropwise to a stirring solution of diethyl 3-fluoro-4-methoxybenzylphosphonate (6 g, 22 mmol) in anhydrous THF (10 mL) at room temperature. Upon completion of the addition, stirring was continued for 45 mins. then tert-butyl 4-oxopiperidine-1-carboxylate (4.8 g, 24.2 mmol) in anhydrous THF (10 mL) was added. The mixture was left to stir for 16 hours before it was quenched with saturated ammonium chloride (250 mL). Extraction with diethyl ether (200 mL) twice and evaporation of the combined organic layer gave a crude oil. Silica gel chromatography eluting with ethyl acetate in hexane (5-15%) gave the title compound (4.7 g, 66%) as a colourless oil.

¹H NMR (CDCl₃) δ: 1.48 (9H, s), 2.31 (2H, m), 2.44 (2H, m), 3.40 (2H, m), 3.50 (2H, m), 3.88 (3H, s), 6.25 (1H, s), 6.80-7.00 (3H, m).

### Description 31

### tert-Butyl 4-(3-fluoro-4-methoxybenzyl)piperidine-1-carboxylate (D31)

A solution of tert-butyl 4-(3-fluoro-4-methoxybenzylidene)piperidine-1-carboxylate (4.7 g, 14.6 mmol) in methanol (400 mL) was allowed to stir under atmospheric pressure of hydrogen at room temperature in the presence of 10% palladium on carbon (0.8 g) for 16 hours. Removal of the catalyst by filtration and evaporation of the filtrate gave a crude oil. Silica gel chromatography eluting with ethyl acetate in hexane (10%) gave the title compound (4.3 g, 99%) as a colourless oil.

¹H NMR (CDCl₃) δ: 1.05-1.20 (2H, m), 1.45 (9H, s), 1.55-1.65 (3H, m), 2.46 (2H, d, J=6 Hz), 2.60-2.70 (2H, m), 3.86 (3H, s), 4.00-4.15 (2H, m), 6.75-6.90 (3H, m).

### Description 32

### 4-(3-Fluoro-4-methoxybenzyl)piperidine (D32)

The title compound was prepared from tert-butyl 4-(3-fluoro-4-methoxybenzyl)piperidine-1-carboxylate using a similar method to Description 20.

### Description 33

### 3,4-Dihydro-5-fluoro-2-methylquinazoline (D33)

A solution of 2-amino-6-fluorobenzylamine (1.1 g, 7.86 mmol) and triethylorthoacetate (1.58 mL, 8.64 mmol) in ethanol (30 mL) was heated at 80° C. for 14 h. The reaction mixture was allowed to cool to room temperature and evaporated in vacuo. The yellow oil was triturated with diethyl ether to give the title compound as white solid (0.74 g, 57%).

Mass spectrum (API⁺): Found 165 (MH⁺). C₉H₉N₂F requires 164.

¹H NMR (CDCl₃) δ: 2.02 (3H, s), 4.67 (2H, s), 6.34-6.71 (2H, m), 7.03-7.12 (1H, m).

### Description 34

### 5-Fluoro-2-methylquinazoline (D34)

To a solution of 3,4-dihydro-5-fluoro-2-methylquinazoline (0.74 g, 4.51 mmol) in chloroform (100 mL) at room temperature was added manganese (IV) oxide (4.0 g, 46.0 mmol) and the reaction mixture stirred at room temperature for 20 h. The reaction mixture was filtered through a plug of celite, washing with dichloromethane. The filtrate was evaporated in vacuo to give the title compound as a yellow solid (0.715 g, 98%).

Mass spectrum (API⁺): Found 163 (MH⁺). C₉H₇N₂F requires 162.

¹H NMR (CDCl₃) δ: 2.92 (3H, s), 7.19-7.27 (1H, m), 7.77-7.83 (2H, m).9.60 (1H, s).

### Description 35

### 2-(2-Methylquinazolin-5-yloxy)ethanol (D35)

To a solution of ethylene glycol (3.05 mL, 55.6 mmol) in N,N-dimethylformamide (50 mL) at room temperature was added sodium hydride (60% dispersion in oil, 0.30 g, 7.50 mmol) portionwise. The reaction mixture was allowed to stir at room temperature for 30 minutes. A solution of 5-fluoro-2-methylquinazoline (2.22 g, 55.6 mmol) in N,N-dimethylformamide (5 mL) was added and the reaction mixture heated at 85° C. for 14 h. The mixture was allowed to cool to room temperature, quenched by the addition of water and concentrated in vacuo. Chromatography of the residue on SiO₂ eluting with 40% ethyl acetate in dichloromethane to ethyl acetate gave the title compound as a yellow solid (0.39 g, 10%).

Mass spectrum (API⁺): Found 205 (MH⁺) C₁₁H₁₂N₂O₂requires 204.

¹H NMR (CDCl₃) δ: 2.87 (3H, s), 4.134.16 (2H, m), 4.13-4.33 (2H, m), 6.88(1H, d, J=8 Hz), 7.50 (1H, d, J=9 Hz), 7.72-7.76 (1H, m), 9.64 (1H, s).

### Description 36

### 5-[2-(Methanesulfonyloxy)ethoxyl-2-methylquinazoline (D36)

To a solution of 2-(2-methylquinazolin-5-yloxy)ethanol (0.330 g, 1.62 mmol) in dichloromethane (20 mL) and triethylamine (0.34 mL, 2.43 mmol) was added methane sulfonyl chloride (0.14 mL, 1.78 mmol) dropwise. The reaction mixture was allowed to stir at room temperature for 2 h. The reaction mixture was diluted with further dichloromethane and partitioned with saturated aqueous NaHCO₃ solution. The organic phase was washed with brine, dried (MgSO₄) and evaporated in vacuo to give the title compound as a cream solid (0.452 g, 99%).

Mass spectrum (ES⁺): Found 283 (MH⁺) C₁₂H₁₄N₂O₄S requires 282.

¹H NMR (CDCl₃) δ: 2.89 (3H, s), 3.10 (3H, s), 4.46-4.48 (2H, m). 4.71-4.73 (2H, m), 6.86 (1H, d, J=8 Hz), 7.55 (1H, d, J=9 Hz), 7.74-7.78 (1H, m), 9.69 (1H, s).

### Description 37

### 8-Chloro-4-hydroxy-5-methoxy-2-(trifluoromethyl)quinoline (D37)

A mixture of 2-chloro-5-methoxyaniline hydrochloride (10.0 g, 0.063 mol) and ethyl (trifluoromethyl)acetoacetate (10.3 mL, 0.070 mol) in polyphosphoric acid (40 mL) was heated to 160° C. under argon for 2 h. Water (200 mL) was added with care and the crude product extracted (EtOAc×2). Chromatography (SiO₂; eluent 20% 60-80° petrol/EtOAc afforded the title compound as a dark yellow solid (3.38 g, 19%).

¹H NMR (400 MHz, CDCl₃) δ: 4.13 (s, 3H), 6.85 (d, 1H), 7.19 (s, 1H), 7.75 (d, 1H), 10.05 (s,1H).

### Description 38

### 4,8-Dichloro-5-methoxy-2-(trifluoromethyl)quinoline (D38)

A mixture of 8-chloro-4-hydroxy-5-methoxy-2-(trifluoromethyl)quinoline (3.38 g, 0.012 mol) and phosphorus pentachloride (2.08 g, 0.01 mol) in phosphorus oxychloride (20 mL) was heated at reflux for 2.5 h. On cooling, water (100 mL) was added with care and the product extracted (CH₂Cl₂×2). The organics were dried (Na₂SO₄) and evaporated in vacuo to give the title compound as a yellow solid (3.19 g, 90%).

¹H NMR (400 MHz, CDCl₃) δ: 4.00 (s, 3H), 6.97 (d, 1H), 7.79 (s, 1H), 7.85 (d, 1H).

### Description 39

### 5-Methoxy-2-(trifluoromethyl)quinoline (D39)

A solution of 4,8-dichloro-5-methoxy-2-(trifluoromethyl)quinoline (2.9 g, 9.8 mmol) in 1M ethanolic potassium hydroxide (100 mL) was hydrogenated over 10% palladium on carbon (50% aqueous paste; 500 mg) at atmospheric temperature and pressure for 18 h. The mixture was filtered through celite, the filtrate evaporated in vacuo and the residue partitioned between CH₂Cl₂ and water. The organics were dried (Na₂SO₄) and evaporated in vacuo. Chromatography (SiO₂; eluent 20% EtOAc/60-80° petrol) afforded the title compound as a yellow solid (850 mg, 38%).

¹H NMR (400 MHz, CDCl₃) δ: 4.04 (s, 3H), 6.96 (d, 1H), 7.70-7.73 (m, 2H), 7.80 (d, 1H), 8.76 (d, 1H).

### Description 40

### 2-(Trifluoromethyl)quinolin-5-ol (D40)

To a solution of 5-methoxy-2-(trifluoromethyl)quinoline (830 mg, 3.65 mmol) in CH₂Cl₂ (20 mL) at 0° C. was added dropwise boron tribromide (1.0 mL, 10.95 mmol). The mixture was stirred under argon whilst allowing to warm to room temp. for 2 h. Water (50 mL) was added with care and the organics separated, dried (Na₂SO₄) and evaporated in vacuo to give a pale yellow oil (570 mg, 73%).

¹H NMR (400 MHz, CDCl₃) δ: 5.97 (br s, 1H), 6.97 (d, 1H), 7.63 (t, 1H), 7.73 (d, 1H), 7.82 (d, 1H), 8.77 (d, 1H).

### Description 41

### 5-(2-Bromoethoxy)-2-(trifluoromethyl)quinoline (D41)

A mixture of 2-(trifluoromethyl)quinolin-5-ol (563 mg, 2.64 mmol), potassium carbonate (1.8 g, 13.0 mmol) and 1,2-dibromoethane (2.3 mL, 26.0 mmol) in methyl ethyl ketone (20 mL) was heated at reflux under argon for 16 h. The solvent was removed in vacuo and the residue partitioned between water and CH₂Cl₂. The organics were dried (Na₂SO₄) and evaporated. Chromatography (SiO₂; eluent 20% to 50% EtOAc/60-80° petrol) afforded the title compound as a buff solid (600 mg, 71%).

¹H NMR (400 MHz, CDCl₃) δ: 3.80 (t, 2H), 4.52 (t, 2H), 6.95 (d, 1H), 7.69-7.74 (m, 2H), 7.84 (d, 1H), 8.82 (d, 1H).

### Description 42

### tert-Butyl 4-(3-pyridin-4-ylbenzyl)piperidine-1-carboxylate (D42)

A mixture of tert-butyl 4-(3-pyridin-4-ylbenzylidene)-piperidine-1-carboxylate (100 mg, 0.29 mmol) and platinum oxide (20 mg) in EtOH (20 ml) was shaken under hydrogen at 50 psi for 48 h. The mixture was filtered and concentrated to dryness under vacuum to afford the title compound as a yellow/brown gum (106 mg, 100%).

Mass spectrum (API+): Found 353 (MH⁺). C₂₂H₂₈N₂O₂ requires 352.

### Description 43

### 4-(3-Pyridin-4-ylbenzyl)piperidine (D43)

A stirred solution of tert-butyl 4-(3-pyridin-4-ylbenzyl)piperidine-1-carboxylate (100 mg, 0.28 g) in DCM (10 ml) was treated with TFA(1 ml). After 18 h the mixture was concentrated to dryness under vacuum and the residue re-dissolved in DCM (1 ml) and eluted through an SCX-2 cartridge which was subsequently eluted successively with DCM, 10% MeOH in DCM, MeOH and finally 2.0 M ammonia in MeOH, concentration of this final fraction afforded the crude title compound as a pale yellow gum, 0.70 g, 97%

Mass spectrum (API+): Found 253 (MH⁺). C₁₇H₂₀N₂ requires 252.

### Description 44

### tert-Butyl 4-(3-cyclopentylcarbamoylbenzyl)piperidine-1-carboxylate (D44)

A solution of tert-butyl 4-(3-cyanobenzyl)piperidine-1-carboxylate (200 mg, 0.63 mmol) was treated with 50% aqueous sodium hydroxide (3 ml), and tetrabutyl ammonium hydrogen sulphate (21 mg, 0.063 mmol) and the mixture heated to reflux. To this mix was added a solution of cyclopentyl bromide (103 mg, 0.69 mmol) in toluene (10 ml) dropwise with stirring. Reflux was continued for 48h then cooled to room temp. The reaction mix was partitioned between ethyl acetate (20 ml) and water (4×20 ml). The organic layer was dried over Na₂SO₄ and evaporated in vacuo. The residue was added to a 5 g pre-packed silica column and eluted from ethyl acetate to give the title compound (202 mg, 83%) as a yellow oil.

Mass spectrum (API⁺): Found 287 (MH-Boc+). C23H34N2O3 requires 386

### Description 45

### N-Cyclopentyl-3-(piperidin-4-ylmethyl)benzamide hydrochloride (D45)

The title compound was prepared from tert-butyl 4-(3-cyclopentylcarbamoylbenzyl)piperidine-1-carboxylate in a manner similar to Description 4, in 97% yield.

Mass spectrum (API⁺): Found 287 (MH+). C18H26N20 requires 286.

### Description 46

### 3-(1-(2-(2-Methylquinolin-5-yloxy)ethyl)piperidin-4-ylidenemethyl)benzoic acid (D46)

Methyl 3-(1-(2-(2-methylquinolin-5-yloxy)ethyl)piperidin-4-ylidenemethyl)benzoate (2.7 mmol) was dissolved in methanol (2 ml) and treated with 2 M aqueous sodium hydroxide solution (6 ml) and the mixture heated to reflux for 18 h. The solution was then allowed to cool to ambient temperature, neutralised (1 M HCl) and the solvent removed in vacuo. The solid obtained was triturated twice (MeOH) to afford the title compound as a solid.

### Description 47

### 1-(3-{1-[2-2-Methylquinolin-5-yloxy)ethyl]piperidin-4-ylidenemethyl}phenyl)-1-piperidin-1-ylmethanone (D47)

3-(1-(2-(2-Methylquinolin-5-yloxy)ethyl)piperidin-4-ylidenemethyl)benzoic acid (0.12 mmol), EDC (23 mg, 0.12 mmol) and HOBT (5 mg) were stirred in dichloromethane (4 ml) and treated with a solution of piperidine (10 mg, 0.12 mmol) in dichloromethane (3 ml). The reaction mixture was shaken for 66 h then partitioned between DCM (10 ml) and saturated NaHCO₃ solution (10 ml). The organic layer was separated, dried (MgSO₄) and the solvent removed in vacuo to afford the title compound as an orange oil (40 mg, 71%).

### Description 48

### 5-{2-[4-(3-Furan-3-ylbenzylidene)piperidin-1-yl]ethoxy}-2-methylquinoline (D48)

Triphenyl phosphine (8 mg, 0.03 mmol), 3-furanylboronic acid (41 mg, 0.37 mmol) and 2M K₂CO₃ (0.7 ml, aq) were added to a solution of 5-{2-[4-(3-iodobenzylidene)piperidin-1-yl]ethoxy}-2-methylquinoline (150 mg, 0.31 mmol) in 1,2-dimethoxyethane (5 ml). The reaction mixture was purged with argon for 5 min. before the addition of palladium acetate (2 mg, 9 μm), followed by an 18 h reflux. The reaction mixture was cooled to room temperature and partioned between water (7 ml) and ethyl acetate (3×10 ml). The organic extracts were combined and the solvent removed in vacuo. Purification of the residue by chromatography, using a 10 g pre-packed silica column eluting with (70-0%) light petroleum (bp 40-60° C.) in ethyl acetate, gave the title compound (84 mg, 64%) as an oil.

Mass spectrum (API⁺): Found 425 (MH⁺). C₂₈H₂₈N₂O₂ requires 424.

### Description 49

### tert-Butyl 4-(3-pyridin-2-ylbenzylidene)piperidine-1-carboxylate (D49)

2-Tributylstannylpyridine (1.058 g, 2.30 mmol) and PdCl₂(PPh₃)₂ (70 mg, 0.1 mmol) were added to a solution tert-butyl 4-(3-iodobenzylidene)piperidine-1-carboxylate (800 mg, 2 mmol) in tetrahydrofuran (15 ml), with stirring under an argon atmosphere at room temperature. The reaction mixture was stirred at reflux for 22 h, under argon, before cooling to room temperature, followed by dilution with diethyl ether (25 ml) and filtration through Kieselguhr. The filtrate was washed with water (4×10 ml); the organic layer collected, dried over Na₂SO₄ and filtered. Solvent removal in vacuo obtained a 2 phase product, which was partitioned between dichloromethane (15 ml) and water (15 ml), with the organic layer being collected and the solvent removed in vacuo. The residue was purified by chromatography using a 20 g pre-packed silica column, eluting with (10-15%) ethyl acetate in light petroleum (bp 40-60° C.), to afford the title compound (170 mg, 25%) as an orange oil.

Mass spectrum (API⁺): Found 251 (MH⁺). C₂₂H₂₆N₂O₂ requires 350.

### Description 50

### 2-(3-(Piperidin-4-ylidenemethyl)phenyl)pyridine dihydrochloride (D50)

The title compound was prepared from tert-butyl 4-(3-pyridin-2-ylbenzylidene)piperidine-1-carboxylate in a manner similar to Description 20, in 89% yield.

Mass spectrum (API⁺): Found 251 (MH⁺). C₁₇H₁₈N₂ requires 250.

### Description 51

### tert-Butyl 4-[3-(3-methyl-1,2,4-oxadiazol-5-yl)benzyl]piperidine-1-carboxylate (D51)

A solution of tert-butyl 4-(3-carbamoylbenzyl)piperidine-1-carboxylate (128 mg, 0.40 mmol) in N,N-dimethylacetamide dimethyl acetal (160 mg, 1.20 mmol) was heated at 125° C. for 1 h. The reaction was cooled to room temp. and azeotroped in 1,4-dioxan (2×5 ml) and evaporated in vacuo to give a brown oil. A solution of hydroxylamine hydrochloride (42 mg, 0.6 mmol) in 5 N sodium hydroxide was treated at room temp. with 70% aqueous acetic acid (0.5 ml) and the mixture stirred for 10 mins. then a solution of the acylamidine in dioxan (0.5 ml) was added and the solution stirred for 0.5 h. The reaction mixture was partitioned between dichloromethane (5 ml) and water (5 ml). The organic layer was separated and added to a 5 g pre-packed silica column and eluted from 50-100% ethyl acetate in petroleum ether (40-60° C.) to give the title compound (150 mg, 100%) as a yellow oil.

¹H NMR (CDCl₃) δ: 1.18 (2H, m), 1.45 (9H, s), 1.62 (2H m), 1.71 (1H, m), 2.48 (3H, m), 2.63 (4H, m), 4.08 (2H, m), 7.36 (1H, m), 7.44 (1H, m), 7.91 (1H, m), 7.95 (1H, m).

### Description 52

### 4-[3-(3-Methyl-1,2,4-oxadiazol-5-yl)benzyl]piperidine (D52)

The title compound was prepared from tert-butyl 4-[3-(3-methyl-1,2,4-oxadiazol-5-yl)benzyl]piperidine-1-carboxylate in a manner similar to Description 20.

Mass spectrum (API+): Found 258 (MH⁺). C₁₅H₁₉N₃O requires 257.

### Description 53

### tert-Butyl 4-(3-cyanobenzyl)piperidine-1-carboxylate (D53)

The title compound was prepared from tert-butyl 4-(3-cyanobenzylidene)piperidine-1-carboxylate in a manner similar to Example 6.

¹H NMR (CDCl₃) δ: 1.14 (2H, m), 1.45 (9H, m), 1.58 (2H, m), 1.67 (1H, m), 2.58 (2H, d J 7), 2.64 (2H, m), 4.11 (2H, m), 7.38 (2H, m), 7.43 (1H, m), 7.50 (1H, m).

### Description 54

### 4-[3-(5-Methyl-1,2,4-oxadiazol-3-yl)benzyl]piperidine (D54)

A mixture of tert-butyl 4-(3-cyanobenzyl)piperidine-1-carboxylate (300 mg, 1 mmol), ammonium acetate (616 mg, 8 mmol), nitroethane (3 ml), and glacial acetic acid (1 ml) was heated at reflux with stirring for 60 h. The reaction mixture was cooled to room temp. and the solvent removed in vacuo. The residue was dissolved in 1 N HCl in ether (10 ml) and stirred at reflux for 3 h, and then evaporated to dryness in vacuo. The residue was partitioned between diethyl ether (5 ml) and water (5 ml). The aqueous layer was basified to pH 10 using 2 N sodium hydroxide and extracted into dichloromethane (2×5 ml). The combined organic phases were dried over Na₂SO₄ and evaporated in vacuo to give the title compound (103 mg, 40%) as a yellow gum.

Mass spectrum (API+): Found 258 (MH⁺). C₁₅H₁₉N₃O requires 257.

### EXAMPLE 1

### 2-Methyl-5-(2-{4-[3-(1-methylpiperidin-4-yl)benzyl]piperidin-1-yl}ethoxy)quinoline hydrochloride (E1)

A mixture of the crude 4-[3-(1-methylpiperidin-4-yl)benzyl]piperidine hydrochloride (0.11 g, 0.36 mmol), 5-(2-bromoethoxy)-2-methylquinoline (0.065 g, 0.24 mmol) and potassium carbonate (0.055 g, 0.40 mmol) in N,N-dimethylformamide (2 mL) was stirred at room temperature for 18 h, and then at 65° C. for 8 h. The mixture was then concentrated to dryness in vacuo and the residue partitioned between water and dichloromethane. The organic phase was dried and concentrated to dryness in vacuo, and the residue subjected to flash chromatography on silica eluting with dichloromethane/methanol/NH₄OH (100:10:1). The title compound was converted to the hydrochloride salt as a buff powder (0.047 g, 40%).

Mass spectrum (API⁺): Found 458 (MH⁺). C₃₀H₃₉N₃O requires 457

¹H NMR(free base) (CDCl₃) δ: 1.31-1.38 (2H, m), 1.53 (1H, m), 1.68 (2H, br d), 1.84 (4H, m), 2.05-2.17 (4H, m), 2.34 (3H, s), 2.45 (1H, m), 2.53 (2H, d), 2.72 (3H, s), 2.95 (2H, t), 3.04 (4H, t), 4.27 (2H,t), 6.80 (1H, d), 6.96 (2H, m), 7.04 (1H, d), 7.22 (2H, m), 7.56 (2H, m), 8.42 (1H, d).

### EXAMPLE 2

### 2-Chloro-5-(1-(2-(2-methylquinolin-5-yloxy)ethyl)piperidin-4-ylmethyl)benzensulfonamide (E2)

The title compound was prepared from 2-chloro-5-(piperidin-4-ylmethyl)benzensulfonamide hydrochloride and 5-(2-bromoethoxy)-2-methylquinoline in a similar manner to Example 1.

Mass spectrum (API⁺): Found 474 (MH⁺). C₂₄H₂₈³⁵ClN₃O₃S requires 473.

¹H NMR (CDCl₃) δ: 1.33 (2H, m), 1.53 (3H, m), 2.13 (2H, m), 2.58 (2H, d, J=7 Hz), 2.73 (3H, s), 2.92 (2H, m), 3.02 (2H, m), 4.27 (2H, m), 5.21 (2H, br s), 6.79 (1H, m), 7.26 (2H, m), 7.43 (1H, d, J=8 Hz), 7.55 (2H, m), 7.88 (1H, m), 8.42 (1H, d, J=9 Hz).

### EXAMPLE 3

### 3-(1-(2-(2-Methylquinolin-5-yloxy)ethyl)piperidin-4-ylmethyl)benzenesulfonamide (E3)

The title compound was prepared from 3-(piperidin-4-ylmethyl)benzensulfonamide hydrochloride and 5-(2-bromoethoxy)-2-methylquinoline in a similar manner to Example 1.

Mass spectrum (API⁺): Found 440 (MH⁺). C₂₄H₂₉N₃O₃S requires 439.

hu 1H NMR (CDCl₃) δ: 1.36 (2H, m), 1.58 (1H, m), 1.62 (2H, m), 2.17 (2H, m), 2.63 (2H, m), 2.73 (3H, s), 2.95 (2H, m), 3.05 (2H, m), 4.27 (2H, m), 4.74 (2H, br s), 6.80 (1H, m), 7.24 (1H, m), 7.36 (1H, m), 7.43 (1H, m), 7.56 (2H, m), 7.72 (1H, m), 7.75 (1H, m), 8.42 (1H, d, J=8.4 Hz).

### EXAMPLE 4

### N,N-Dimethyl-3-(1-(2-(2-methylquinolin-5-yloxy)ethyl)piperidin-4-ylmethyl)benzenesulfonamide (E4)

A solution of 3-(1-(2-(2-methylquinolin-5-yloxy)ethyl)piperidin-4-ylmethyl) benzenesulfonamide (0.005 g, 0.00011 mol) in dry tetrahydrofuran (10 mL) was treated with a 60% suspension of sodium hydride in oil (0.01 g, 0.00025 mol) and the mixture stirred at 20° C. for 0.5 h. Iodomethane (0.032 g, 0.00023 mol) was then added to the mixture and stirring continued for a further 24 h. The solvent was removed in vacuo and the residue partitioned between ethyl acetate (10 mL) and water (3×10 mL). The organic layer was dried (Na₂SO₄) and evaporated in vacuo to give the title compound (0.053 g, 100%) as an oil.

Mass spectrum (API⁺): Found 468 (MH⁺). C₂₆H₃₃N₃O₃S requires 467.

¹H NMR (CDCl₃) δ: 1.33 (2H, m), 1.62 (3H, m), 2.15 (2H, m), 2.63 (2H, m), 2.71 (9H, m), 2.94 (2H, m), 3.05 (2H, m), 4.27 (2H, m), 6.79 (1H, m), 7.25 (1H, m), 7.41 (2H, m), 7,57 (4H, m), 8.42 (1H, d, J=8 Hz).

### EXAMPLE 5

### 3-(1-(2-(2-Methylquinolin-5-yloxy)ethyl)piperidin-4-ylidenemethyl)benzonltrile (E5)

A mixture of 5-(2-bromoethoxy)-2-methylquinoline (1 g, 3.8 mmol), 4-(3-cyanobenzylidene)piperidine hydrochloride (0.88 g, 3.8 mmol), and potassium carbonate (1.6 g, 11.3 mmol) in N,N-dimethylformamide (30 mL) was stirred at 90° C. for 3 h. The reaction mixture was cooled and partitioned between ethyl acetate (25 mL) and water (3×20 mL). The organic layer was dried (Na₂SO₄) and evaporated in vacuo. Chromatography of the residue on SiO₂ eluting with 30-100% ethyl acetate in hexane gave the title compound (0.98 g, 68%) as an oil.

Mass spectrum (API⁺): Found 384 (MH⁺). C₂₅H₂₅N₃O requires 383.

¹H NMR (CDCl₃) δ: 2.43-2.49 (2H, m), 2.49-2.51 (2H, m), 2.63-2.66 (2H, m), 2.73 (3H, s), 2.74-2.77 (2H, m), 2.99 (2H, t J 6), 4.30 (2H, t J 6), 6.25 (1H, s), 6.81 (1H, d, J=7 Hz), 7.25-7.27 (1H, m), 7.40-7.41 (2H, m), 7.47-7.50 (2H, m), 7.84-7.62 (2H, m), 8.44 (1H, d J=8 Hz).

### EXAMPLE 6

### 3-(1-(2-(2-Methylquinolin-5-yloxy)ethyl)piperidin-4-ylmethyl)benzonitrile (E6)

A mixture of 3-(1-(2-(2-methylquinolin-5-yloxy)ethyl)piperidin-4-ylidenemethyl)benzonitrile (0.9 g, 2.3 mmol) and 10% palladium on charcoal (paste) (0.1 g) in methanol (100 mL) was stirred at 20° C., under an atmosphere of hydrogen (1 atm) for 2 h. The reaction mixture was filtered through celite and the evaporated in vacuo to give a residue. Chromatography of the residue on SiO₂ eluting with 30-100% ethyl acetate in hexane gave the title compound (0.72 g, 81%) as an solid.

Mass spectrum (API⁺): Found 386 (MH⁺). C₂₅H₂₇N₃O requires 385.

hu 1H NMR (CDCl₃) δ: 1.32-1.39 (2H, m), 1.61-1.69 (2H, m), 1.69 (1H, br s), 2.15-2.17 (2H, m), 2.58 (2H, d J 7), 2.72 (3H, m), 2.92 (2H, t J 6), 3.02-3.06 (2H, m), 4.27 (2H, t J 6), 6.79 (1H, d J 8), 7.23-7.26 (1H, m), 7.37-7.38 (2H, m), 7.43 (1H, s), 7.48-7.61 (3H, m), 8.42 (1H, d, J 8).

### EXAMPLE 7

### Methyl 3-(1-(2-(2-methylquinolin-5-yloxy)ethyl)piperidin-4-ylmethyl)benzoate (E7)

The title compound was prepared from methyl 3-(1-(2-(2-methylquinolin-5-yloxy)ethyl)piperidin-4-ylidenemethyl)benzoate in a similar manner to Example 6.

Mass spectrum (API⁺): Found 419 (MH⁺). C₂₆H₃₀N₂O₃ requires 418.

¹H NMR (CDCl₃) δ: 1.35 (2H, m), 1.48 (1H, m), 1.64 (2H, m), 2.14 (2H, m), 2.60 (2H d, J=7 Hz), 2.72 (3H, s), 2.92 (2H, m), 3.03 (2H, m), 3.91 (3H, s), 4.27 (2H, m), 6.79 (1H, dd, J=7, 1 Hz), 7.24 (1H, d, J=8 Hz), 7.33 (2H, m), 7.56 (2H, m), 7.83 (2H, m), 8.42 (1H, d, J=9 Hz).

### EXAMPLE 8

### 3-(1-(2-(2-Methylquinolin-5-yloxy)ethyl)piperidin-4-ylmethyl)benzamide (E8)

The title compound was prepared from 3-(piperidin4-ylmethyl)benzamide hydrochloride and 5-(2-bromoethoxy)-2-methylquinoline in a similar manner to Example 5.

Mass spectrum (API⁺): Found 404 (MH⁺). C₂₅H₂₉N₃O₂ requires 403.

¹H NMR (CDCl₃) δ: 1.40 (2H, m), 1.56 (1H, m), 1.65 (2H, m), 2.17 (2H, m), 2.59 (2H d, J=7 Hz), 2.72 (3H, s), 2.96 (2H, m), 3.09 (2H, m), 4.29 (2H, m), 5.85 (1H, br s), 6.23 (1H, br s), 6.79 (1H, m), 7.23-7.36 (3H, m), 7.52-7.65 (4H, m), 8.42 (1H, d, J=9 Hz).

### EXAMPLE 9

### N,N-Dimethyl-3-(1-(2-(2-methylquinolin-5-yloxy)ethyl)piperidin-4-ylmethyl)benzamide (E9)

The title compound was prepared from 3-(1-(2-(2-methylquinolin-5-yloxy)ethyl)piperidin-4-ylmethyl)benzamide in a similar manner to Example 4.

Mass spectrum (API⁺): Found 432 (MH⁺). C₂₇H₃₃N₃O₂ requires 431.

¹H NMR (CDCl₃) δ: 1.33 (2H, m), 1.52 (1H, m), 1.65 (2H, m), 2.14 (2H, m), 2.56 (2H, m), 2.70 (9H, m), 2.95 (2H, m), 3.05 (2H, m), 4.27 (2H, m), 6.87 (1H, m), 7.18-7.30 (4H, m), 7.31-7.66 (3H, m), 8.35 (1H, d, J=9 Hz)

### EXAMPLE 10

### 1-(3-(1-(2-(2-Methylquinolin-5-yloxy)ethyl)piperidin-4-ylmethyl)phenyl)ethanone (E10)

A solution of diethyl methylphosphonate (0.076 g, 0.0052 mol) in dry tetrahydrofuran (10 mL) was cooled to −78° C. (CO₂/acetone) and treated with a 2.5 M solution of butyllithium (0.21 mL, 0.00052 mol) with stirring under argon. The mixture was stirred at −78° C. under argon for 1 h. A solution of 3-(1-(2-(2-methylquinolin-5-yloxy)ethyl)piperidin-4-ylmethyl)benzonitrile (0.2 g, 0.00052 mol) in dry tetrahydrofuran (10 mL) was added dropwise over 0.1 h. The mixture was allowed to warm up to −5° C. over 0.75 h and treated with a 1M solution of lithium aluminium hydride (1.6 mL, 0.0016 mol). The mixture was warmed to 20° C. and stirred for 0.5 h and treated with a 5 M solution of sulfuric acid dropwise until effervescence ceased, and the mix stirred at 20° C. for 18 h. This mixture was treated with saturated sodium bicarbonate solution to pH 9 and filtered through celite. The filtrate was partitioned between dichloromethane (20 mL) and water (3×10 mL). The organic layer was dried (Na₂SO₄) and evaporated in vacuo. Chromatography of the residue on SiO₂ eluting from 0-10% methanol in ethyl acetate gave the title compound (0.017 g, 8%) as an oil.

Mass spectrum (API⁺): Found 403 (MH⁺). C₂₆H₃₀N₂O₂ requires 402.

¹H NMR (CDCl₃) δ: 1.35 (2H, m), 1.47 (1H, m), 1.65 (2H, m), 2.14 (2H, m), 2.61 (5H, m), 2.72 (3H, s), 2.92 (2H, m), 3.03 (2H, m), 4.27 (2H, m), 6.79 (1H, m), 7.24 (1H, d, J=9 Hz), 7.36 (2H, m), 7.56 (2H, m), 7.77 (2H, m), 8.42 (1H, d, J=9 Hz).

### EXAMPLE 11

### 3-(1-(2-(2-Methylquinolin-5-yloxy)ethyl)piperidin-4-ylidenemethyl)bromobenzene (E11)

The title compound was prepared from 4-(3-bromobenzylidene)piperidine hydrochloride and 5-(2-bromoethoxy)-2-methylquinoline in a similar manner to Example 5.

Mass spectrum (API⁺): Found 437 (MH⁺). C₂₄H₂₅⁷⁹BrN₂O requires 436.

¹H NMR (CDCl₃) δ: 2.43 (2H, m), 2.52 (2H, m), 2.64 (2H, m), 2.74 (5H, m), 2.98 (2H, m), 4.30 (2H, m), 6.23 (1H, s), 6.81 (1H, dd, J=7 Hz, 1 Hz), 7.11 (1H, m), 7.17 (1H, t, J=8 Hz), 7.27 (1H, d, J=8 Hz), 7.33 (2H, m), 7.58 (2H, m), 8.44 (1H, d, J=8 Hz).

### EXAMPLE 12

### 3-(1-(2-(2-Methylquinolin-5-yloxy)ethyl)piperidin-4-ylidenemethyl)methoxybenzene (E12)

The title compound was prepared from 4-(3-methoxybenzylidene)piperidine hydrochloride and 5-(2-bromoethoxy)-2-methylquinoline in a similar manner to Example 5.

Mass spectrum (API⁺): Found 389 (MH⁺). C₂₅H₂₈N₂O₂ requires 388.

¹H NMR (CDCl₃) δ: 2.43 (2H, m), 2.57 (2H, m), 2.63 (2H, m), 2.74 (5H, m), 2.98 (2H, m), 3.80 (3H, s), 4.30 (2H, m), 6.28 (1H, s), 6.78 (4H, m), 7.24 (2H, m), 7.57 (2H, m), 8.44 (1H, d, J=8 Hz).

### EXAMPLE 13

### 3-(1-(2-(2-Methylquinolin-5-yloxy)ethyl)piperidin-4-ylmethyl)methoxybenzene (E13)

The title compound was prepared from 3-(1-(2-(2-methylquinolin-5-yloxy)ethyl)piperidin-4-ylidenemethyl)methoxybenzene in a similar manner to Example 6.

Mass spectrum (API⁺): Found 391 (MH⁺). C₂₅H₃₀N₂O₂ requires 390.

¹H NMR (CDCl₃) δ: 1.42 (2H, m), 1.57 (1H, m), 1.69 (2H, m), 2.22 (2H, m), 2.53 (2H, d, J=7 Hz), 2.72 (3H, s), 2.99 (2H, m), 3.11 (2H, m), 3.79 (3H, s), 4.32 (2H, m), 6.71 (3H, m), 6.79 (1H, m), 7.22 (2H, m), 7.58 (2H, m), 8.41 (1H, d, J=9 Hz).

### EXAMPLE 14

### 3-(1-(2-(2-Methylquinolin-5-yloxy)ethyl)piperidin-4-ylmethyl)phenyl methanesulponate (E14)

Stage 1

A mixture of 3-(1-(2-(2-methylquinolin-5-yloxy)ethyl)piperidin-4-ylmethyl)methoxybenzene (0.25 g, 0.00064 mol), 48% aqueous hydrobromic acid (1 mL) and acetic acid (1 mL) was heated at 100° C. for 6 h, and then evaporated in vacuo to give (0.37 g) an oil.

Stage 2

Half of this material was dissolved in pyridine (3 mL) and treated with methansulfonic anhydride (63 mg, 0.00036 mol) and the mixture stirred at 20° C. for 3 h. The mixture was partitioned between dichloromethane (15 mL) and water (5×20 mL). The organic layer was dried (Na₂SO₄) and evaporated in vacuo to give the title compound (0.063 g, 46%) as an oil.

Mass spectrum (API⁺): Found 455 (MH⁺). C₂₅H₃₀N₂O₄S requires 454.

¹H NMR (CDCl₃) δ: 1.36 (2H, m), 1.57 (1H, m), 1.66 (2H, m), 2.17 (2H, m), 2.58 (2H, d, J=7 Hz), 2.73 (3H, s), 2.95 (2H, m), 3.07 (2H, m), 3.14 (3H, s), 4.28 (2H, 6.79 (1H, d, J=7 Hz), 7.10 (2H, m), 7.28 (3H, m), 7.57 (2H, m), 8.42 (1H, d, J=9 Hz).

### EXAMPLE 15

### O-[3-(1-(2-(2-Methylquinolin-5-yloxy)ethyl)piperidin-4-ylmethyl)phenyl] acetate (E15)

The title compound was prepared in a similar manner to Example 14.

Mass spectrum (API⁺): Found 419 (MH⁺). C₂₆H₃₀N₂O₃ requires 418.

¹H NMR (CDCl₃) δ: 1.59 (3H, m), 1.74 (2H, m), 2.28 (3H, s), 2.42 (2H, m), 2.57 (2H, d, J=7 Hz), 2.74 (3H, s), 3.22 (2H, m), 3.39 (2H, m), 4.40 (2H, m), 6.80 (1H, d, J=8 Hz), 6.87 (1H, d, J=2 Hz), 6.93 (1H, dd, J=8 Hz, 2 Hz), 7.00 (1H, d, J=8 Hz), 7.26 (2H, m), 7.56 (1H, t, J=8 Hz), 7.66 (1H, d, J=9 Hz), 8.40 (1H, d, J=9 Hz).

### EXAMPLE 16

### 3-(1-(2-(2-Methylquinolin-5-yloxy)ethyl)piperidin-4-ylmethyl)benzylamine (E16)

The title compound was prepared in a similar manner to Example 10.

Mass spectrum (API⁺): Found 390 (MH⁺). C₂₅H₃₁N₃O requires 389.

¹H NMR (CDCl₃) δ: 1.38 (2H, m), 1.56 (1H, m), 1.67 (2H, m), 2.16 (2H, m), 2.54 (2H, m), 2.72 (3H, s), 2.82 (2H, m), 2.95 (2H, m), 3.07 (2H, m), 3.85 (2H, s), 4.28 (2H, m), 6.79 (1H, m), 7.03 (1H, d, J=8 Hz), 7.11 (2H, m), 7.24 (2H, m), 7.57 (2H, m), 8.42 (1H, d, J=9 Hz).

### EXAMPLE 17

### N-(3-(1-(2-(2-Methylquinolin-5-yloxy)ethyl)piperidin-4-ylmethyl)benzyl)methanesulfonamide (E17)

The title compound was prepared from 3-(1-(2-(2-methylquinolin-5-yloxy)ethyl)piperidin-4-ylmethyl)benzylamine in a similar manner to Stage 2-Example 14.

Mass spectrum (API⁺): Found 468 (MH⁺). C₂₆H₃₃N₃O₃S requires 467.

hu 1H NMR (CDCl₃) δ: 1.33 (2H, m), 1.54 (1H, m), 1.63 (2H, m), 2.14 (2H, m), 2.54 (2H, d, J=7 Hz), 2.72 (3H, s), 2.87 (3H, s), 2.93 (2H, m), 3.03 (2H, m), 4.27 (4H, m), 4.85 (1H, m), 6.79 (1H, m), 7.10 (2H, m), 7.16 (1H, m), 7.25 (2H, m), 7.56 (2H, m), 8.42 (1H, d, J=9 Hz).

### EXAMPLE 18

### N-(3-1-(2-2-Methylquinolin-5-yloxy)ethyl)piperidin-4-ylmethyl)benzyl)acetamide (E18)

The title compound was prepared from 3-(1-(2-(2-methylquinolin-5-yloxy)ethyl)piperidin-4-ylmethyl)benzylamine and acetic anhydride in a similar manner to Stage 2-Example 14.

Mass spectrum (API⁺): Found 432 (MH⁺). C₂₇H₃₃N₃O₂ requires 431.

¹H NMR (CDCl₃) δ: 1.37 (2H, m), 1.56 (1H, m), 1.66 (2H, m), 2.02 (3H, s), 2.18 (2H, m), 2.54 (2H, d, J=7 Hz), 2.72 (3H, s), 2.97 (2H, m), 3.09 (2H, m), 4.29 (2H, m), 4.41 (2H, m), 5.77 (1H, bs), 6.79 (1H, d, J=7 Hz), 7.06 (2H, m), 7.10 (1H, m), 7.24 (2H, m), 7.56 (2H, m), 8.42 (1H, d, J=9 Hz).

### EXAMPLE 19

### N-Isopropyl-N-methyl-3-(1-[2-(2-methylquinolin-5-yloxy)ethyl]piperidin-4-ylmethyl}benzamide (E19)

The title compound was prepared from 3-{1-[2-(2-methylquinolin-5-yloxy)ethyl]piperidin-4-ylmethyl}benzoic acid and N-methylisopropylamine in a manner similar to Description 46, in 79% yield.

Mass spectrum (API⁴): Found 460 (MH⁺). C₂₉H₃₇N₃O₂ requires 459. ¹H NMR (CDCl₃) δ: 1.10-1.24 (6H, m), 1.35 (2H, m), 1.55 (1H, m), 1.66 (2H, m), 2.13 (2H, m), 2.56 (2H, d, J 7), 2.72 (3H, s), 2.76 and 2.92 (5H, m, rotameric), 3.03 (2H, m), 3.94 and 4.96 (1H, m, rotameric), 4.27 (2H, t, J 6), 6.79 (1H, m), 7.16 (3H, m), 7.27 (2H, m), 7.56 (2H, m), 8.43 (1H, d, J 9).

### EXAMPLE 20

### N-Methyl-3-{1-[2-(2-methylquinolin-5-yloxy)ethyl]piperidin-4-ylmethyl}-N-phenylbenzamide (E20)

The title compound was prepared from 3-{1-[2-(2-methylquinolin-5-yloxy)ethyl]piperidin-4-ylmethyl}benzoic acid and N-methyl aniline in a manner similar to Description 46, in 45% yield.

Mass spectrum (API⁺): Found 494 (MH⁺). C₃₂H₃₅N₃O₂ requires 493.

hu 1H NMR (CDCl₃) δ: 1.15 (2H, m), 1.26 (1H, m), 1.37 (2H, m), 2.04 (2H, m), 2.34 (2H, d, J 7), 2.73 (3H, s), 2.91 (2H, t, J 6), 2.96 (2H, m), 3.49 (3H, s), 4.27 (2H, t, J 6), 6.80 (1H, dd, J 7 and 1), 6.96-7.24 (10H, m), 7.57 (2H, m), 8.44 (1H, d, J 9).

### EXAMPLE 21

### N,N-Diethyl-3-{1-[2-(2-methylquinolin-5-yloxy)ethyl]piperidin-4-ylmethyl}benzamide (E21)

The title compound was prepared from 3-{1-[2-(2-methylquinolin-5-yloxy)ethyl]piperidin-4-ylmethyl)benzoic acid and N,N-diethylamine in a manner similar to Description 46, in 84% yield.

Mass spectrum (API⁺): Found 460 (MH⁺). C₂₉H₃₇N₃O₂ requires 459.

¹H NMR (CDCl₃) δ: 1.10 (3H, br s), 1.25 (3H, br s), 1.34 (2H, m), 1.55 (1H, m), 1.65 (2H, m), 2.12 (2H, m), 2.55 (2H, d, J 7), 2.72 (3H, s), 2.92 (2H, t, J 6), 3.03 (2H, m), 3.24 (2H, brs), 3.54 (2H, brs), 4.26 (2H, t, J 6), 6.79 (1H, m), 7.17 (3H, m), 7.27 (2H, m), 7.56 (2H, m), 8.43 (1H, d, J 9).

### EXAMPLE 22

### 1-(3-{1-[2-(2-Methylquinolin-5-yloxy)ethyl]piperidin-4-ylmethyl}phenyl)-1-pyrrolidin-1-ylmethanone (E22)

The title compound was prepared from 3-{1-[2-(2-methylquinolin-5-yloxy)ethyl]piperidin-4-ylmethyl}benzoic acid and pyrrolidine in a manner similar to Description 46, in 80% yield.

Mass spectrum (API⁺): Found 458 (MH⁺). C₂₉H₃₅N₃O₂ requires 457.

¹H NMR (CDCl₃) δ: 1.35 (2H, m), 1.56 (1H, m), 1.66 (2H, m), 1.87 (2H, m), 1.95 (2H, m), 2.13 (2H, m), 2.56 (2H, d, J 7), 2.72 (3H, s), 2.92 (2H, t, J 6), 3.03 (2H, m), 3.41 (2H, t, J 7), 3.64 (2H, t, J 7), 4.27 (2H, t, J 6), 6.79 (1H, m), 7.18 (1H, d, J 7), 7.23 -7.34 (4H, m), 7.56 (2H, m), 8.43 (1H, d, J 9).

### EXAMPLE 23

### 3-{4-[2-(2-Methylquinolin-5-yloxy)ethyl]piperazin-1-ylmethyl}benzonitrile (E23)

A mixture of 2-methyl-5-(2-piperazin-1-ylethoxy)quinoline (0.04 g, 0.15 mmol) and 3-cyanobenzaldehyde (0.021 g, 0.15 mmol) in 1,2-dichloroethane (5 mL) was treated with sodium triacetoxyborohydride (47 mg, 0.22 mmol) and stirred at 20° C. under an atmosphere of argon for 24 h. The mixture was then treated with saturated aqueous NaHCO₃ (20 mL) and the organic layer separated and purified directly by chromatography on silica (ethyl acetate to 10% methanol/ethyl acetate), to afford the title compound (0.032 g, 52%) as a solid.

Mass spectrum (API⁺): Found 387 (MH⁺). C₂₄H₂₆N₄O requires 386.

The following examples (2444) were prepared in a similar manner to Example E23.

### EXAMPLE 24

### 5-{2-[4-(2,3-Dichlorobenzyl)piperazin-1-yl]ethoxy}-2-methylquinoline (E24)

Mass spectrum (API⁺): Found 430 (MH⁺). C₂₃H₂₅³⁵Cl₂N₃O requires 429.

### EXAMPLE 25

### 5-{2-[4-(Benzo[1,3]dioxol-4-ylmethyl)piperazin-1-yl]ethoxy}-2-methylquinoline (E25)

Mass spectrum (API⁺): Found 406 (MH⁺). C₂₄H₂₇N₃O₃ requires 405.

### EXAMPLE 26

### 5-{2-[4-(3,5-Dichlorobenzyl)piperazin-1-yl]ethoxy}-2-methylquinoline (E26)

Mass spectrum (API⁺): Found 430 (MH⁺). C₂₃H₂₅³⁵Cl₂N₃O requires 429.

### EXAMPLE 27

### 5-{2-[4-(2,5-Dichlorobenzyl)piperazin-1-yl]ethoxy}-2-methylquinoline (E27)

Mass spectrum (API⁺): Found 430 (MH⁺). C₂₃H₂₅³⁵Cl₂N₃O requires 429.

### EXAMPLE 28

### 5-{2-[4-(3,4-Dichlorobenzyl)piperazin-1-yl]ethoxy}-2-methylquinoline (E28)

Mass spectrum (API⁺): Found 430 (MH⁺). C₂₃H₂₅³⁵Cl₂N₃O requires 429.

### EXAMPLE 29

### 5-{2-[4-(4-Methoxynaphth-1-ylmethyl)piperazin-1-yl]ethoxy}-2-methylquinoline (E29)

Mass spectrum (API⁺): Found 442 (MH⁺). C₂₈H₃₁N₃O₂ requires 441.

### EXAMPLE 30

### 5-{2-[4-(Quinolin-8-ylmethyl)piperazin-1-yl]ethoxy}-2-methylquinoline (E30)

Mass spectrum (API⁺): Found 413 (MH⁺). C₂₆H₂₈N₄O requires 412.

### EXAMPLE 31

### 5-(2-{4-[3,5-Dichloro-2-(2-morpholin-4-ylethoxy)benzyl]piperazin-1-yl}ethoxy)-2-methylquinoline (E31)

Mass spectrum (API⁺): Found 559 (MH⁺). C₂₉H₃₆³⁵Cl₂N₄O₃ requires 558.

### EXAMPLE 32

### 5-{2-[4-(3-Fluorobenzyl)piperazin-1-yl]ethoxy}-2-methylquinoline (E32)

Mass spectrum (API⁺): Found 380 (MH⁺). C₂₃H₂₆FN₃O requires 379.

### EXAMPLE 33

### 5-{2-[4-(1H-Indol-5-ylmethyl)piperazin-1-yl]ethoxy}-2-methylquinoline (E33)

Mass spectrum (API⁺): Found 401 (MH⁺). C₂₅H₂₆N₄O requires 400.

### EXAMPLE 34

### 2-Methyl-5-(2-{4-[3-(2-morpholin-4-ylethoxy)benzyl]piperazin-1-yl}ethoxy)quinoline (E34)

Mass spectrum (API⁺): Found 491 (MH⁺). C₂₉H₃₈N₄O₃ requires 490.

### EXAMPLE 35

### 2-Methyl-5-(2-{4-[3-(2-pyrrolidin-1-ylethoxy)benzyl]piperazin-1-yl}ethoxy)quinoline (E35)

Mass spectrum (API⁺): Found 475 (MH⁺). C29H₃₈N₄O₂ requires 474.

### EXAMPLE 36

### N,N-Dimethyl-[2-(3-{4-[2-(2-methylquinolin-5-yloxy)ethyl]piperazin-1-ylmethyl}phenoxy)ethyl]amine (E36)

Mass spectrum (API⁺): Found 449 (MH⁺). C₂₇H₃₈N₄O₂ requires 448.

### EXAMPLE 37

### 2-Methyl-5-{2-[4-(3-pyrimidin-5-ylbenzyl)piperazin-1-yl]ethoxy}quinoline (E37)

Mass spectrum (API⁺): Found 440 (MH⁺). C₂₇H₂₉N₅O requires 439.

### EXAMPLE 38

### 5-{2-[4-(Quinolin-6-ylmethyl)piperazin-1-yl]ethoxy}-2-methylquinoline (E38)

Mass spectrum (API⁺): Found 413 (MH⁺). C₂₆H₂₈N₄O requires 412.

### EXAMPLE 39

### 5-Fluoro-6-4-[2-(2-methylquinolin-5-yloxy)ethyl]piperazin-1-ylmethyl}-3H-benzooxazol-2-one (E39)

Mass spectrum (API⁺): Found 437 (MH⁺). C₂₄H₂₅FN₄O₃ requires 436.

### EXAMPLE 40

### 6-{4-[2-(2-Methylquinolin-5-yloxy)ethyl]piperazin-1-ylmethyl}-3,4-dihydro-2H-isoquinolin-1-one (E40)

Mass spectrum (API⁺): Found 431 (MH⁺). C₂₆H₃₀N₄O₂ requires 430.

### EXAMPLE 41

### 5-{2-[4-(2,2-Difluorobenzo[1,3]dioxol-5-ylmethyl)piperazin-1-yl]ethoxy}-2-methyl-quinoline (E41)

Mass spectrum (API⁺): Found 442 (MH⁺). C₂₄H₂₅F₂N₃O₃ requires 441.

### EXAMPLE 42

### 5-{2-[4-(3-Chlorobenzyl)piperazin-1-yl]ethoxy)-2-methylquinoline (E42)

Mass spectrum (API⁺): Found 396 (MH⁺). C₂₃H₂₆³⁵ClN₃O requires 395.

### EXAMPLE 43

### 5-{2-[4-(3-Methoxybenzyl)piperazin-1-yl]ethoxy}-2-methylquinoline (E43)

Mass spectrum (API⁺): Found 392 (MH⁺). C₂₄H29N₃O₂ requires 391.

### EXAMPLE 44

### 5-{2-[4-(1H-Indol-7-ylmethyl)piperazin-1-yl]ethoxy}-2-methylquinoline (E44)

Mass spectrum (API⁺): Found 401 (MH⁺). C₂₅H₂₈N₄O requires 400.

### EXAMPLE 45

### 5-{2-[4-(3-Fluoro-4-methoxybenzyl)piperidin-1-yl]ethoxy}-2-methylquinoline (E44)

The title compound was prepared from 4-(3-fluoro-4-methoxybenzyl)piperidine in a similar manner to Example 5.

Mass spectrum (API⁺): Found 409 (MH⁺). C₂₅H₂₉FN₂O₂ requires 408.

¹H NMR (CDCl₃) δ: 1.20-1.40 (2H, m), 1.41-1.60 (1H, m), 1.61-1.70 (2H, m), 2.05-2.20 (2H, m), 2.57 (2H, d, J=11 Hz), 2.73 (3H, s), 2.92 (2H, t, J=10 Hz), 2.97-3.10 (2H, m), 3.86 (3H, s), 4.27 (2H, t, J=10 Hz), 6.70-6.95 (4H, m), 7.25 (1H, d, J=13 Hz), 7.50-7.65 (2H, m), 8.43 (1H, d, J=13 Hz).

### EXAMPLE 46

### 5-(2-{4-[3-(1-Methylpiperidin-4-yl)benzyl]piperidin-1-yl}ethoxy)-2-trifluoromethylquinoline (E46)

The title compound was prepared from 5-(2-bromoethoxy)-2-trifluoromethylquinoline in a similar manner to Example 1.

Mass spectrum (API⁺): Found 512 (MH⁺). C₃₀H₃₆F₃N₃O requires 511.

¹H NMR (CDCl₃) δ: 1.34 (2H, m), 1.52 (1H, m), 1.68 (2H, brd), 1.79-1.89 (4H, m), 2.08-2.17 (4H, m), 2.36 (3H, s), 2.47 (1H, m), 2.53 (2H, d), 2.94 (2H, t), 3.01 (4H, d), 4.31 (2H, t), 6.94-7.00 (3H, m), 7.06 (1H, d), 7.21 (1H, t), 7.69 (2H, m), 7.82 (1H, d), 8.73 (1H, d).

### EXAMPLE 47

### 2-Methyl-5-(2-{4-[3-(1-methylpiperidin-4-yl)benzyl]piperidin-1-yl)ethoxy)quinazoline (E47)

The title compound was prepared from 5-[2-(methanesulfonyloxy)ethoxy]-2-methylquinazoline in a similar manner to Example 1.

Mass spectrum (API⁺): Found 459 (MH⁺). C₂₉H₃₈N₄O requires 458.

¹H NMR (CDCl₃) δ: 1.34 (2H, m), 1.56 (1H, m), 1.64 (2H, brd), 1.84 (4H, m), 2.14 (4H, m), 2.37 (3H, s), 2.45 (1H, m), 2.52 (2H, d), 2.88 (3H, s), 2.92 (2H, t), 3.03 (4H, m), 4.30 (2H, t), 6.84 (1H, d), 6.97 (2H, m), 7.06 (1H, d), 7.20 (1H, t), 7.47 (1H, d), 7.74 (1H, t), 9.63 (1H, s).

### EXAMPLE 48

### 2-Methyl-5-(2-(4-[3-(piperidin-1-yl)benzyl]-piperidin-1-yl}ethoxy)quinoline (E48)

To a stirred solution of 2-methyl-5-(2-{4-[3-(1-methylpiperidin-1-yl)benzyl]piperidin-1-yl}ethoxy)quinoline (70 mg, 0.15 mmol) and diisopropylethylamine (58 mg, 0.45 mmol) in 1,2-dichloroethane (5 ml) was added, dropwise, 1-chloroethyl chloroformate (32 mg, 0.23 mmol) and the resultant mixture heated to reflux for 18 h. After concentration to dryness in vacuum, the residue was subjected to flash chromatography on silica gel eluting with 5% MeOH in DCM. The purified intermediate was dissolved in MeOH (5 ml) and heated to reflux for 6 hrs, then concentrated to dryness under vacuum to afford the title compound, which was converted to the hydrochloride salt and isolated as a tan solid, 40 mg (56%).

Mass spectrum (API+): Found 444 (MH⁺). C₂₉H₃₇N₃O requires 443.

¹H NMR(free base) (CDCl₃) δ: 1.46 (3H, m), 1.55 (2H, m), 1.66 (2H, br d), 2.05 (2H, br d), 2.24 (4H, m), 2.55(2H, d; J=7 Hz), 2.75 (3H, s), 2.99 (4H, m), 3.10 (2H, m), 3.66 (2H, m), 4.31 (2H, t), 6.81 (1H, d; J=8 Hz), 7.06 (3H, m), 7.23 (2H, m), 7.61 (2H, 8.43 (1H, d; J=9 Hz)

### EXAMPLE 49

### 2-Methyl-5-(2-{4-[3-(1-acetylpiperidin-1-yl)benzyl]piperidin-1-yl}ethoxy)quinoline (E49)

A stirred solution of 2-methyl-5-(2-{4-[3-(piperidin-1-yl)benzyl]piperidin-1-yl}ethoxy)quinoline (40 mg, 0.09 mmol) and pyridine (21 mg, 0.27 mmol) in DCM (5 ml) was treated with a solution of acetic anhydride (46 mg, 0.45 mmol) in DCM (1 ml). After 2 h the mixture was concentrated to dryness under vacuum and the residue subjected to chromatography on silica gel eluting with 2% MeOH in DCM to afford the title compound, which was converted to the hydrochloride salt and isolated as a pale buff solid, 27 mg (61%).

Mass spectrum (API+): Found 486 (MH⁺). C₃₁H₃₉N₃O₂ requires 485.

¹H NMR(free base) (CDCl₃) δ: 1.63 (3H, m), 1.75 (3H, m), 1.83-1.92 (2H, br t), 2.13 (3H, s), 2.48-2.65 (4H, m), 2.73 (3H, s), 3.15-3.27 (3H, m), 3.38 (3H, m), 3.67 (2H, m), 3.94 (1H, br d), 4.51 (2H, br s), 4.79 (1H, br s), 6.83 (1H, d; J=8 Hz), 6.95-7.04 (3H, m), 7.23 (2H, m), 7.56 (1H, t), 7.62 (1H, d), 8.37 (1H d; J=9 Hz)

### EXAMPLE 50

### 2-Methyl-5-(2-{4-[3-(1-methylsulphonylpiperidin-1-yl)benzyl]piperidin-1-yl}ethoxy)quinoline (E50)

A stirred solution of 2-methyl-5-(2-{4-[3-(piperidin-1-yl)benzyl]piperidin-1-yl}ethoxy)quinoline (40 mg, 0.09 mmol) and diisopropylethylamine (35 mg, 0.27 mmol) in DCM (5 ml) was treated with a solution of methanesulphonyl chloride (31 mg, 0.27 mmol) in DCM (1 ml). After 3 h the mixture was concentrated to dryness under vacuum and the residue re-dissolved in DCM (1 ml) and eluted through an SCX-2 cartridge which was subsequently eluted successively with DCM, 10% MeOH in DCM, MeOH and finally 5% aq. ammonia in MeOH. Concentration of this final fraction afforded the title compound, which was converted to the hydrochloride salt and isolated as a pale buff solid, 35 mg (79%).

Mass spectrum (API+): Found 522 (MH⁺). C₃₀H₃₉N₃O₃S requires 521.

¹H NMR(free base) (CDCl₃) δ: 1.31-1.36 (2H, m), 1.54 (2H, m), 1.64 (2H, br d), 1.80-1.86 (2H, m ), 1.93 (2H, m), 2.14 (2H, m), 2.52 (2H, d), 2.57 (1H, m), 2.72 (3H, s), 2.76(1H, m), 2.81 (3H, s), 2.93 (2H, t), 3.04(2H, br d), 3.94 (2H, br d), 4.27 (2H, t), 6.78 (1H, d; J=9 Hz), 6.91-7.02 (3H, m), 7.23 (2H, m), 7.52-7.60 (2H, m), 8.42 (1H d; J=8.5 Hz)

### EXAMPLE 51

### 2-Methyl-5-(2-{4-[3-pyridin-4-ylbenzyl]piperidin-1-yl}ethoxy)quinoline (E51)

The title compound was prepared 4-(3-pyridin-4-ylbenzyl)piperidine and 5-(2-bromoethoxy)-2-methylquinoline in a similar manner to Example 1.

Mass spectrum (API+): Found 438 (MH⁺). C₂₉H₃₁N₃O requires 437.

¹H NMR (free base) (CDCl₃) δ: 1.34-1.43 (2H, m), 1.62 (1H, m), 1.68 (2H, brd), 2.15 (2H, m), 2.2 (2H, d; J=7 Hz), 2.72 (3H, s), 2.92 (2H, t), 2.81 (3H, s), 3.06(2H, br d), 4.27 (2H, t), 6.78 (1H, d; J=7.5 Hz), 7.40 (2H, m), 7.49-7.67 (5H, m), 8.41 (1H d; J=8.5 Hz), 8.6 (2H, d: J=6 Hz)

### EXAMPLE 52

### 5-{2-[4-(4-Fluoro-3-pyridin-4-ylbenzylidene)piperidin-1-yl]ethoxy}-2-methylquinoline (E52)

The title compound was prepared using analogous routes and intermediates to those used to prepare Example 51.

Mass spectrum (API⁺): Found 454 (MH⁺). C₂₉H₂₈N₃OF requires 453.

¹H NMR (400 MHz, CDCl₃) δ: 2.45 (2H, t J 5.2), 2.54 (2H, t J 5.6), 2.65 (2H, t J 5.6), 2,72 (3H, s), 2.75 (2H, t J 5.2), 2.99 ( 2H, t J 5.6), 6.29 (1H, s), 6.81 (1H, d J 7.6), 7.13 (1H, t J 10.4), 7.23-7.28 (3H, m), 7.46 (2H, m), 7.53-7.60 (2H, m), 8.43 (1H, d J 8.8), 8.66 (2H, d J 4.4).

### EXAMPLE 53

### 5-{2-[4-(4-Fluoro-3-pyridin-4-ylbenzyl)piperidin-1-yl]ethoxy}-2-methylquinoline (E53)

The title compound was prepared from 5-{2-[4-(4-fluoro-3-pyridin-4-ylbenzylidene)piperidin-1-yl]ethoxy}-2-methylquinoline according to the method of Example 6.

Mass spectrum (API⁺): Found 456 (MH⁺). C₂₉H₃₀N₃OF requires 455.

¹H NMR (400 MHz, CDCl₃) δ: 1.34-1.43 (2H, m), 1.57-1.62 (1H, br), 1.69 (2H, d J 12.8), 2.15 (2H, t J 11.68), 2.63 (2H, d J 7.0), 2.72 (3H, s), 2.93 (2H, t J 5.8), 3.04 (2H, d J 11.5), 4.27 (2H, t J 5.8), 6.78 (1H, d J 7.4), 7.21-7.26 (2H, m), 7.37-7.40 (2H, m), 7.46-7.60 (6H, m), 8.42 (1H, d J 8.56), 8.65 (2H, d J 5.8).

### EXAMPLE 54

### 5-(2-{4-[4-Fluoro-3-(1-methylpiperidin-4-yl)benzyl]piperidin-1-yl}ethoxy)-2-methylquinoline (E54)

The title compound was prepared using analogous routes and intermediates to those used to prepare Example 1.

Mass spectrum (API⁺): Found 476 (MH⁺). C₃₀H₃₈N₃OF requires 475.

¹H NMR (400 MHz, CDCl₃) δ: 1.32 (2H, m), 1.45 (1H, m br), 1.63 (2H, d J12.4), 1.82-1.89 (4H, m), 2.13 (4H, t J 11.6), 2.36 (3H, s), 2.48 (2H, d J 6.8), 2.72 (3H, s), 2.83 (1H, m), 2.93 (2H, t J 5.6), 3.02 (4H, m), 4.27 (2H, t J 6.0), 6.79 (1H, d J 7.2), 6.90 (2H, m), 7.24 (1H, d J 8.4), 7.52-7.60 (2H, m), 8.43 (1 h, d J 8.4).

### EXAMPLE 55

### 5-{2-[4-(4-Fluoro-3-piperidin-4-ylbenzyl)piperidin-1-yl]ethoxy}-2-methylquinoline (E55)

The title compound was prepared from 5-(2-{4-[4-fluoro-3-(1-methylpiperidin-4-yl)benzyl]piperidin-1-yl}ethoxy)-2-methylquinazoline according to the method of Example 48.

Mass spectrum (API⁺): Found 462 (MH⁺). C₂₉H₃₆N₃OF requires 461.

¹H NMR (400 MHz, CDCl₃) δ: 1.82-2.05 (13H, br m), 2.59 (2H, d J 6.5), 2.73 (3H, s), 2.92 (1H, br, m), 3.00-3.06 (2H, t J 12.4 br), 3.09-3.14 (4H, m, br), 4.69 (2H, t, br), 5.57 (1H, br), 6.86 (1H, d J 7.6), 6.92-6.96 (1H, m), 7.05 (1H, d J 6.8), 7.26 (2H, m), 7.56 (1H, t J 7.6), 7.66 (1H, d J 8.4), 8.40 (1H, d J 8.5).

### EXAMPLE 56

### 5-(2-(4-[4-Fluoro-3-(1-methylpiperidin-4-yl)benzyl]piperidin-1-yl}ethoxy)-2-methylquinazoline (E56)

The title compound was prepared using analogous routes and intermediates to those used to prepare Example 47.

Mass spectrum (API⁺): Found 477 (MH⁺). C₂₉H₃₇N₄OF requires 476.

¹H NMR (400 MHz, CDCl₃) δ: 1.29-1.35 (2H, m br), 1.49 (1H, m br), 1.63 (2H, d J 12.4), 1.83-1.92 (4H, m br), 2.12-2.22 (4H, m br), 2.40 (3H, s), 2.48 (2H, d J 6.8), 2.85 (1H, m br), 2.88 (3H, s), 2.94 (2H, t J 5.6), 3.05 (4H, t J 13.6), 4.30 (2H, t J 5.6), 6.85 (1H, d J 8.0), 6.90 (2H, m), 6.99 (1H, d J 7.6), 7.48 (1H, d J 8.4), 7.74 (1H, t J 8.3), 9.63 (1H, s).

### EXAMPLE 57

### 5-[2-(4-(Biphenyl-3-ylmethylene)piperidin-1-yl)ethoxy]-2-methylquinoline (E57)

The title compound was prepared from 5-{2-[4-(3-bromobenzylidene)piperidin-1-yl]ethoxy}-2-methylquinoline and phenyl boronic acid according to the method of Description 1.

Mass spectrum (API⁺): Found 435 (MH⁺). C₃₀H₃₀N₂O requires 434.

hu 1H NMR (CDCl₃) δ: 2.46 (2H, m), 2.65 (4H, m), 2.73 (3H, s), 2.76 (2H, m), 2.98 (2H, t J 6), 4.30 (2H, t J6), 6.36 (1H, s), 6.81 (1H, d J8), 7.19 (1H, m), 7.24 (2H, m), 7.34-7.45 (5H, m), 7.58 (4H, m), 8.44 (1H, d J 9).

### EXAMPLE 58

### 5-[2-(4-(Biphenyl-3-ylmethyl)piperidin-1-yl)ethoxy]-2-methylquinoline (E58)

The title compound was prepared from 5-[2-(4-(biphenyl-3-ylmethylene)piperidin-1-yl)ethoxy]-2-methylquinoline in a manner similar to Example 6.

Mass spectrum (API⁺): Found 437 (MH⁺). C₃₀H₃₂N₂O requires 436.

¹H NMR (CDCl₃) δ: 1.39 (2H, m), 1.59(1H, m), 1.70 (2H, m), 2.14(2H, m), 2.61 (2H, d J 7), 2.72 (3H, s), 2.92 (2H, t J 6), 3.04 (2H, m), 4.27 (2H, t J 6), 6.78 (1H, dd J 7 and 2), 7.12 (1H, m), 7.23 (2H, m), 7.33-7.46 (5H, m), 7.57(4H, m), 8.42 (1H, d J 9).

### EXAMPLE 59

### 5-(2-[4-3-Bromobenzylidene)piperidin-1-yl]ethoxy}-2-methylquinazoline (E59)

The title compound was prepared from 4-(3-bromobenzylidene)piperidine and 5-[2-(methanesulfonyloxy)ethoxy]-2-methylquinazoline in a manner similar to Example 5.

Mass spectrum (API⁺): Found 438 (MH⁺). C₂₃H₂₄⁷⁹BrN₃O requires 437.

hu 1H NMR (CDCl₃) δ: 2.42 (2H, m), 2.52 (2H, m), 2.63 (2H, m), 2.75 (2H, m), 2.88 (3H, s), 2.99 (2H, m), 4.46 (2H, m), 6.23 (1H, bs), 6.86 (1H, d J 8), 7.11 (1H, m), 7.17 (1H, m), 7.32 (2H, m), 7.56 (1H, d J 9), 7.74 (1H, m), 9.64 (1H, s).

### EXAMPLE 60

### 5-{2-[4-(3-Iodobenzylidene)piperidin-1-yl]ethoxy}-2-methylquinoline (E60)

The title compound was prepared from 4-(3-iodobenzylidene)piperidine using analogous routes and intermediates to those used to prepare Example 11.

Mass spectrum (API⁺): Found 485 (MH⁺). C₂₄H₂₅IN₂O requires 484.

¹H NMR (CDCl₃) δ: 2.42 (2H, m), 2.52 (2H, m), 2.63 (2H, m), 2.74 (5H, m), 2.98 (2H, m), 4.30 (2H, m), 6.20 (1H, bs), 6.80 (1H, d J 7), 7.04 (1H, t J 8), 7.15 (1H, m), 7.26 (1H, m), 7.57 (4H, m), 8.44 (1H, d J9).

### EXAMPLE 61

### 5-{2-[4-(3-Furan-3-ylbenzyl)piperidin-1-yl]ethoxy}-2-methylquinoline (E61)

The title compound was prepared from 5-{2-[4-(3-furan-3-ylbenzylidene)piperidin-1-yl]ethoxy}-2-methylquinoline in a manner similar to Example 6, in 11% yield.

Mass spectrum (API⁺): Found 427 (MH⁺). C₂₈H₃₀N₂O₂ requires 426.

¹H NMR (CDCl₃) δ: 1.35 (2H, m), 1.58 (1H, m), 1.69 (2H, m), 2.14 (2H, m), 2.57 (2H, d, J 7), 2.72 (3H, s), 2.92 (2H, t, J 6), 3.03 (2H, m), 4.27 (2H, t, J 6), 6.70 (1H, m), 6.79 (1H, m), 7.05 (1H, m), 7.22-7.31 (3H, m), 7.47-7.60 (4H, m), 7.78 (1H, m), 8.42 (1H, d J 9).

### EXAMPLE 62

### 2-Methyl-5-{2-[4-(3-pyridin-3-ylbenzylidene)piperidin-1-yl]ethoxy}quinoline (E62)

The title compound was prepared from 5-{2-[4-(3-iodobenzylidene)piperidin-1-yl]ethoxy}-2-methylquinoline and 3-pyridylboronic acid in a manner similar to Description 1, in 18% yield.

Mass spectrum (API⁺): Found 436 (MH⁺). C₂₉H29N₃O requires 435.

¹H NMR (CDCl₃) δ: 2.47 (2H, m), 2.60 (2H, m), 2.66 (2H, m), 2.73 (3H, s), 2.77 (2H, m), 2.99 (2H, t, J 6), 4.31 (2H, t, J 6), 6.37 (1H, s), 6.81 (1H, m), 7.25 (1H, m), 7.35 (3H, m), 7.42 (2H, m), 7.57 (2H, m), 7.86 (1H, m), 8.44 (1H, d, J 8), 8.59 (1H, dd, J 5 and 2), 8.85 (1H, m).

### EXAMPLE 63

### 2-Methyl-5-{2-[4-(3-pyridin-3-ylbenzyl)piperidin-1-yl]ethoxy}quinoline (E63)

The title compound was prepared from 2-methyl-5-{2-[4-(3-pyridin-3-ylbenzylidene)piperidin-1-yl]ethoxy}quinoline in a manner similar to Example 6, in 65% yield.

Mass spectrum (API⁺): Found 438 (MH⁺). C₂₉H₃₁N₃O requires 437.

¹H NMR (CDCl₃) δ: 1.38 (2H, m), 1.61 (1H, m), 1.70 (2H, m), 2.15 (2H, m), 2.63 (2H, d J 7), 2.72 (3H, s), 2.93 (2H, t, J 6), 3.05 (2H, m), 4.27 (2H, t, J 6), 6.79 (1H, m), 7.21 (2H, m), 7.37 (4H, m), 7.57 (2H, m), 7.86 (1H, m), 8.42 (1H, d, J 9), 8.58 (1H, dd, J 5 and 2), 8.84 (1H, m).

### EXAMPLE 64

### 2-Methyl-5-{2-[4-(3-pyridin-2-ylbenzylidene)piperidin-1-yl]ethoxy}quinoline (E64)

The title compound was prepared from 2-(3-(piperidin-4-ylidenemethyl)phenyl)pyridine and 5-(2-bromoethoxy)-2-methylquinoline in a manner similar to Example 5, in 34% yield.

Mass spectrum (API⁺): Found 436 (MH⁺). C₂₉H₂₉N₃O requires 435.

¹H NMR (CDCl₃) δ: 2.46 (2H, m), 2.63 (4H, m), 2.72 (3H, s), 2.76 (2H, m), 2.98 (2H, t, J 6), 4.30 (2H, t, J 6), 6.39 (1H, s), 6.81 (1H, m), 7.25 (3H, m), 7.41 (1H, t, J8), 757 (2H, m), 7.73 (2H, m), 7.82 (2H, m), 8.44 (1H, d, J 9), 8.69 (1H, m).

### EXAMPLE 65

### 2-Methyl-5-2-[4-(3-pyridin-2-ylbenzyl)piperidin-1-yl]ethoxy}quinoline (E65)

The title compound was prepared from 2-methyl-5-{2-[4-(3-pyridin-2-ylbenzylidene)piperidin-1-yl]ethoxy}quinoline in a manner similar to Example 6, in 100% yield.

Mass spectrum (API⁺): Found 438 (MH⁺). C₂₉H₃₁N₃O requires 437.

¹H NMR (CDCl₃) δ: 1.39 (2H, m), 1.63 (1H, m), 1.70 (2H, m), 2.14 (2H, m), 2.64 (2H, m), 2.64 (2H, d, J 7), 2.72 (3H, s), 2.92 (2H, t, J 6), 3.03 (2H, m), 4.27 (2H, t, J 6), 6.79 (1H, dd, J 7 and 1), 7.22 (3H, m), 7.38 (1H, m), 7.56 (2H, m), 7.72-7.81 (4H, m), 8.42 (1H, d, J 9), 8.69 (1H, m).

### EXAMPLE 66

### 1-Cyclohexyl-1-(3-{1-[2-(2-methylquinolin-5-yloxy)ethyl]piperidin-4-ylmethyl}phenyl)methanone (E66)

The title compound was prepared from 1-cyclohexyl-1-(3-{1-[2-(2-methylquinolii yloxy)ethyl]piperidin-4-ylidenemethyl}phenyl)methanone in a manner similar to Example 67.

Mass spectrum (API⁺): Found 471 (MH⁺). C₃₁H₃₈N₂O₂ requires 470.

¹H NMR (CDCl₃) δ 1.38 (4H, m), 1.50 (3H, m), 1.65 (3H, m), 1.74 (1H, m), 1.86 (4H, m), 2.15 (m), 2.61 (2H, d J 7), 2.72 (3H, s), 2.93 (2H, m), 3.04 (2H, m), 3.24 (1H, m), 4.27 (2H, m), 6.79 (dd J 7 and 1), 7.24 (1H, d J 8), 7.34 (2H, m), 7.56 (2H, m), 7.71 (1H, m), 7.75 (1H, dd J 8 and 8.42 (1H, d J 8).

### EXAMPLE 67

### 1-(3-{1-[2-(2-Methylquinolin-5-yloxy)ethyl]piperidin-4-ylmethyl}phenyl)-1-phenyl-methanone (E67)

A solution of 3-{1-[2-(2-methylquinolin-5-yloxy)ethyl]piperidin-4-ylmethyl{benzonitrile (100 mg, 0.26 mmol) in benzene (25 ml) was added dropwise to a 1 M solution of phenyl magnesium bromide in tetrahydrofuran (2.6 ml, 2.6 mmol) with stirring, at room temperature, under an argon atmosphere. The reaction mixture was heated at reflux, under argon, for 16 h before being cooled to room temperature and partitioned between ethyl acetate (50 ml) and saturated aqueous NH₄Cl (30 ml). The organic layer was collected and the solvent removed in vacuo. The residue was stirred in 2 M HCl (75 ml) for 8 h, before neutralising with 6 M NaOH to pH 7. The product was extracted into dichloromethane (2×30 ml) and the combined organic phases were dried over Na₂SO₄, filtered and the solvent removed in vacuo. The residue was purified by chromatography on a 5 g pre-packed silica column, eluting from (20-90%) ethyl acetate in light petroleum (bp 40-60° C.), to afford the title compound (25 mg, 21%) as a yellow oil.

Mass spectrum (API⁺): Found 465 (MH⁺). C₃₁H₃₂N₂O₂ requires 464.

¹H NMR (CDCl₃) δ: 1.31-1.40 (2H, m), 1.57 (1H, m), 1.67 (2H, m), 2.14 (2H, m), 2.62 (2H, d, J 7), 2.72 (3H, s), 2.92 (2H, t, J 6), 3.04 (2H, m), 4.27 (2H, t, J 6), 6.79 (1H, m), 7.24 (1H, d, J 9), 7.37 (2H, m), 7.49 (2H, m), 7.54-7.61 (5H, m), 7.80 (2H, m), 8.43 (1H, d, J 9).

### EXAMPLE 68

### 1-(3-{1-2-(2-Methylquinolin-5-yloxy)ethyl]piperidin-4-ylmethyl{phenyl)propan-1-one (E68)

The title compound was prepared from 3-{1-[2-(2-methylquinolin-5-yloxy)ethyl]piperidin-4-ylmethyl}benzonitrile and ethyl magnesium bromide in a manner similar to Example 67, in 7% yield.

Mass spectrum (API⁺): Found 417 (MH⁺). C₂₇H₃₂N₂O₂ requires 416.

¹H NMR (CDCl₃) δ: 1.23 (3H, t, J=7 Hz), 1.36 (2H, m), 1.55-1.66 (3H, m), 2.13 (2H, m), 2.61 (2H, d, J=7 Hz), 2.72 (3H, s), 2.92 (2H, t, J=6Hz), 2.97-3.05 (4H, m), 4.27 (2H, t, J=6 Hz), 6.79 (1H, m), 7.24 (1H, d, J=9 Hz), 7.35 (2H, m), 7.56 (2H, m), 7.77 (2H, m), 8.42 (1H, d, J=8 Hz).

### EXAMPLE 69

### 2-Methyl-5-(2-{4-[3-(3-methyl-1,2,4-oxadiazol-5-yl)benzyl]piperidin-1-yl}ethoxy)quinoline (E69)

The title compound was prepared from 4-[3-(3-methyl-1,2,4-oxadiazol-3-yl)benzyl]piperidine manner similar to Example 5.

Mass spectrum (API⁺): Found 443 (MH⁺). C₂₇H₃₀N₄O₂ requires 442.

¹H NMR (CDCl₃) δ: 1.37 (2H, m), 1.65 (3H, m), 2.15 (2H, m), 2.48 (3H, m), 2.63 (2H, d J 7), (3H, s), 2.93 (2H, m), 3.04 (2H, m), 4.27 (2H, m), 6.79 (1H, d J 7), 7.24 (1H, d J 9), 7.36 (1H, 7.43 (1H, m), 7.56 (2H, m), 7.93 (2H, m), 8.42 (1H, d J 9).

### EXAMPLE 70

### 2-Methyl-5-(2-{4-[3-(5-methyl-1,2,4-oxadiazol-3-yl)benzyl]piperidin-1-yl)ethoxy)quinoline (E70)

The title compound was prepared from 4-[3-(5-methyl-1,2,4-oxadiazol-3-yl)benzyl]piperidine manner similar to Example 5.

Mass spectrum (API⁺): Found 443 (MH⁺). C₂₇H₃₀N₄O₂ requires 442.

¹H NMR (CDCl₃) δ: 1.37 (2H, m), 1.61 (1H, m), 1.68 (2H, m), 2.14 (2H, m), 2.61 (2H, d J 7), (3H, s) 2.72 (3H, s), 2.92 (2H, m), 3.03 (2H, m), 4.27 (2H, m), 6.79 (1H, d J 7), 7.26 (2H, m), (1H, m), 7.56 (2H, m), 7.88 (2H, m), 8.42 (1H d J 8).

### EXAMPLE 71

### N-Cyclopentyl-3-{1-[2-(2-methylquinolin-5-yloxy)ethyl]piperidin-4-ylmethyl}benzamide (E71)

The title compound was prepared from N-cyclopentyl-3-(piperidin-4-ylmethyl)benzamide and 5-(2-bromoethoxy)-2-methylquinoline in a manner similar to Example 1, in 13% yield.

Mass spectrum (API⁺): Found 472 (MH⁺). C₃₀H₃₇N₃O₂ requires 471.

¹H NMR (CDCl₃) δ: 0.85 (2H, m), 1.40 (2H, m), 1.49 (2H, m), 1.55-1.78 (5H, m), 2.09 (2H, m), 2.19 (2H, m), 2.59 (2H, d, J 7), 2.72 (3H, s), 2.98 (2H, m), 3.09 (2H, m), 4.30 (2H, m), 4.40 (1H, m), 6.06 (1H, d, J 7), 6.79 (1H, m), 7.24 (2H, m), 7.31 (1H, t, J 8), 7.51-7.61 (4H, m), 8.41 (1H, d, J 9).

### EXAMPLE 72

### 1-(3-{1-[2-(2-Methylquinolin-5-yloxy)ethyl]piperidin-4-ylmethyl}phenyl)-1-(piperidin-1-yl)methanone (E72)

The title compound was prepared from 1-(3-{1-[2-(2-methylquinolin-5-yloxy)ethyl]piperidin-4-ylidenemethyl}phenyl)-1-(piperidin-1-yl)methanone in a manner similar to Example 6, in 18% yield.

Mass spectrum (API⁺): Found 472 (MH⁺). C₃₀H₃₇N₃O₂ requires 471.

hu 1H NMR (CDCl₃) δ: 0.86 (2H, m), 1.32 (2H, m), 1.54 (1H, m), 1.66 (6H, m), 2.13 (2H, m), 2.56 (2H, d, J 7), 2.72 (3H, s), 2.92 (2H, t, J 6), 3.03 (2H, m), 3.33 (2H, m), 3.70 (2H, m), 4.27 (2H, t, J6), 6.79 (1H, dd, J7 and 1), 7.16-7.31 (5H, m), 7.56 (2H, m), 8.43 (1H, d J 9).

### EXAMPLE 73

### 3-1-[2-(2-Methylquinolin-5-yloxy)ethyl]piperidin-4-ylmethyl}-N-propylbenzamide (E73)

The title compound was prepared from 3-{1-[2-(2-methylquinolin-5-yloxy)ethyl]piperidin-4-ylmethyl}benzoic acid and n-propylamine in a manner similar to Description 46, in 79% yield.

Mass spectrum (API⁺): Found 446 (MH⁺). C₂₈H₃₅N₃O₂ requires 445.

¹H NMR (CDCl₃) δ: 0.99 (3H, t, J 7), 1.32-1.39 (2H, m), 1.57 (1H, m), 1.64 (4H, m), 2.12 (2H, m), 2.58 (2H, d, J 7), 2.72 (3H, s), 2.92 (2H, t, J 6), 3.03 (2H, m), 3.42 (2H, m), 4.26 (2H, t, J6), 6.16 (1H, br s), 6.79 (1H, m), 7.24 (2H, m), 7.32 (1H, t, J 8), 7.52-7.60 (4H, m), 8.42 (1H, d, J 9).

### EXAMPLE 74

### N-Isopropyl-3-{1-[2-(2-methylquinolin-5-yloxy)ethyl]piperidin-4-ylmethyl}benzamide (E74)

The title compound was prepared from 3-{1-[2-(2-methylquinolin-5-yloxy)ethyl]piperidin-4-ylmethyl}benzoic acid and isopropylamine in a manner similar to Description 46, in 82% yield.

Mass spectrum (API⁺): Found 446 (MH⁺). C₂₈H₃₆N₃O₂ requires 445.

¹H NMR (CDCl₃) δ: 1.27 (6H, d, J=7 Hz), 1.29-1.39 (2H, m), 1.57 (1H, m), 1.64 (2H, m), 2.13 (2H, td, J=12 and 2 Hz), 2.58 (2H, d, J=7 Hz), 2.72 (3H, s), 2.92 (2H, t, J=6 Hz), 3.03 (2H, m), 4.28 (3H, m), 5.92 (1H, d, J=7 Hz), 6.79 (1H, m), 7.24 (2H, m), 7.31 (1H, t, J=8 Hz), 7.51-7.60 (4H, m), 8.42 (1H, d, J=9 Hz).

### EXAMPLE 75

### 3-{1-[2-(2-Methylquinolin-5-yloxy)ethyl]piperidin-4-ylmethyl}-N-phenylbenzamide (E75)

The title compound was prepared from 3-{1-[2-(2-methylquinolin-5-yloxy)ethyl]piperidin-4-ylmethyl}benzoic acid and aniline in a manner similar to Description 46, in 59% yield.

Mass spectrum (API⁺): Found 480 (MH⁺). C₃₁H33N₃O₂ requires 479.

¹H NMR (CDCl₃) δ: 1.31-1.40 (2H, m), 1.58 (1H, m), 1.65 (2H, m), 2.13 (2H, m), 2.60 (2H, d, J 7), 2.71 (3H, s), 2.92 (2H, t, J 6), 3.02 (2H, m), 4.26 (2H, t, J 6), 6.79 (1H, m), 7.15 (1H, t, J 8), 7.24 (1H, d, J 9), 7.32-7.39 (4H, m), 7.57 (2H, m), 7.66 (4H, m), 7.92 (1H, s), 8.43 (1H, d, J8).

### EXAMPLE 76

### N-Isopropyl-N-methyl-3-{1-[2-(2-methylquinolin-5-yloxy)ethyl]piperidin-4-ylmethyl}benzamide (E76)

The title compound was prepared from 3-{1-[2-(2-methylquinolin-5-yloxy)ethyl]piperidin-4-ylmethyl)benzoic acid and N-methylisopropylamine in a manner similar to Description 46, in 79% yield.

Mass spectrum (API⁺): Found 460 (MH⁺). C₂₉H₃₇N₃O₂ requires 459.

¹H NMR (CDCl₃) δ: 1.10-1.24 (6H, m), 1.35 (2H, m), 1.55 (1H, m), 1.66 (2H, m), 2.13 (2H, m), 2.56 (2H, d, J 7), 2.72 (3H, s), 2.76 and 2.92 (5H, m, rotameric), 3.03 (2H, m), 3.94 and 4.96 (1H, m), 4.27 (2H, t, J 6), 6.79 (1H, m), 7.16 (3H, m), 7.27 (2H, m), 7.56 (2H, m), 8.43 (1H, d, J 9).

### EXAMPLE 77

### N-Methyl-3-{1-[2-(2-methylquinolin-5-yloxy)ethyl]piperidin-4-ylmethyl}-N-phenylbenzamide (E77)

The title compound was prepared from 3-{1-[2-(2-methylquinolin-5-yloxy)ethyl]piperidin-4-ylmethyl}benzoic acid and N-methyl aniline in a manner similar to Description 46, in 45% yield.

Mass spectrum (API⁺): Found 494 (MH⁺). C₃₂H₃₅N₃O₂ requires 493.

¹H NMR (CDCl₃) δ: 1.15 (2H, m), 1.26 (1H, m), 1.37 (2H, m), 2.04 (2H, m), 2.34 (2H, d, J 7), 2.73 (3H, s), 2.91 (2H, t, J 6), 2.96 (2H, m), 3.49 (3H, s), 4.27 (2H, t, J 6), 6.80 (1H, dd, J 7 and 1), 6.96-7.24 (10H, m), 7.57 (2H, m), 8.44 (1H, d, J 9).

### EXAMPLE 78

### N,N-Diethyl-3-1-[2-(2-methylquinolin-5-yloxy)ethyl]piperidin-4-ylmethyl}benzamide (E78)

The title compound was prepared from 3-{1-[2-(2-methylquinolin-5-yloxy)ethyl]piperidin-4-ylmethyl)benzoic acid and N,N-diethylamine in a manner similar to Description 46, in 84% yield.

Mass spectrum (API⁺): Found 460 (MH⁺). C₂₉H₃₇N₃O₂ requires 459.

¹H NMR (CDCl₃) δ: 1.10 (3H, br s), 1.25 (3H, br s), 1.34 (2H, m), 1.55 (1H, m), 1.65 (2H, m), 2.12 (2H, m), 2.55 (2H, d, J 7), 2.72 (3H, s), 2.92 (2H, t, J 6), 3.03 (2H, m), 3.24 (2H, br s), 3.54 (2H, br s), 4.26 (2H, t, J 6), 6.79 (1H, m), 7.17 (3H, m), 7.27 (2H, m), 7.56 (2H, m), 8.43 (1H, d, J 9).

### EXAMPLE 79

### 1-(3-{1-[2-(2-Methylquinolin-5-yloxy)ethyl]piperidin-4-ylmethyl}phenyl)-1-pyrrolidin-1-ylmethanone (E79)

The title compound was prepared from 3-{1-[2-(2-methylquinolin-5-yloxy)ethyl]piperidin-4-ylmethyl}benzoic acid and pyrrolidine in a manner similar to Description 46, in 80% yield.

Mass spectrum (API⁺): Found 458 (MH⁺). C₂₉H₃₅N₃O₂ requires 457.

hu 1H NMR (CDCl₃) δ: 1.35 (2H, m), 1.56 (1H, m), 1.66 (2H, m), 1.87 (2H, m), 1.95 (2H, m), 2.13 (2H, m), 2.56 (2H, d, J 7), 2.72 (3H, s), 2.92 (2H, t, J 6), 3.03 (2H, m), 3.41 (2H, t, J 7), 3.64 (2H, t, J 7), 4.27 (2H, t, J 6), 6.79 (1H, m), 7.18 (1H, d, J 7), 7.23-7.34 (4H, m), 7.56 (2H, m), 8.43 (1H, d, J 9).

1. A compound of formula (I) or a pharmaceutically acceptable salt thereof: 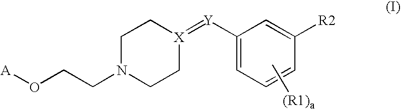
wherein
A is optionally substituted phenyl, indolyl, quinolinyl, quinazolinyl, indazolyl or isoquinolinyl;
X is carbon, Y is CH and ═ is a double bond; or X is CH, Y is CH₂ or oxygen and ═ is a single bond; or X is nitrogen, Y is CH₂ and ═ is a single bond;
R1 is halogen, cyano, nitro, C₁₋₆alkyl, haloC₁₋₆alkyl, C₁₋₆alkoxy, C₃₋₇heterocyclylC₁₋₆alkyl, C₃₋₇heterocyclyl C₁₋₆alkoxy;
a is 0, 1, 2, 3 or 4;
R2 is either:
halogen, —CN, an optionally substituted C₃₋₇cycloalkyl, an optionally substituted aryl or an optionally substituted C-linked 3-7 membered heterocyclic group;
or
a group -(Z)b-B wherein:
(i) Z is oxygen, CH₂, C═O, SO₂ or C═N—OR3 wherein R3 is hydrogen or C₁₋₆alkyl; b is 1, 2, or 3; and B is hydrogen, C₁₋₆alkyl, C₃₋₇cycloalkyl, C₁₋₆alkoxy or NR4R5 wherein R4 and R5 are independently hydrogen, C₁₋₆alkyl, C₃₋₇cycloalkyl, C₁₋₆alkanoyl, fluoroC₁₋₆alkanoyl, C₁₋₆alkylsulfonyl, fluoroC₁₋₆alkylsulfonyl, carbamoyl or C₁₋₆alkylcarbamoyl, or R4 and R5, together with the nitrogen atom to which they are attached, form part of an optionally substituted 3 to 7 membered heterocyclic group;
or
(ii) Z is oxygen, CH or CH₂, b is 1, and B forms the rest of an aryl or a C₃₋₇heterocyclic group fused to the phenyl ring;
excluding
1-[2-[2-(phenylmethyl)phenoxy]ethyl]-4-[(2,3,4-trimethoxyphenyl)methyl]-piperazine and pharmaceutically acceptable salts thereof, and
N-[4-[2-[4-[(3,4-dimethoxyphenyl)methyl]-1-piperazinyl]ethoxy]phenyl]-methanesulfonamide and pharmaceutically acceptable salts thereof.

2. A compound as claimed in claim 1, wherein A is quinolinyl or quinazolinyl.

3. A compound as claimed in claim 2, wherein A is 5-(2-methyl)quinolinyl or 5-(2-methyl)quinazolinyl.

4. A compound as claimed in claim 1 wherein R1 is fluoro.

5. A compound as claimed in claim 1, wherein b is 0, 1 or 2.

6. A compound as claimed in claim 1, which is any of Examples 1-79 or a pharmaceutically acceptable salt thereof.

7. A process for the preparation of a compound of formula (I) as defined in claim 1 or a pharmaceutically acceptable salt thereof, which process comprises:
(a) the coupling of a compound of formula (II): 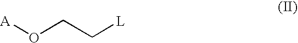
wherein A is as defined for formula (I) and L is a leaving group,
with a compound of formula (III): 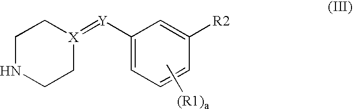
wherein a, X, Y, R1, R2 and are as defined for formula (I);
or
(b) for a compound wherein X is nitrogen, the coupling of a compound of formula (IV): 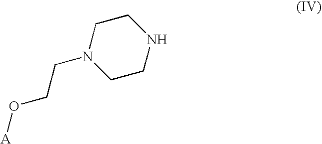
wherein A has the same meanings as for formula (I), and a compound of formula (V): 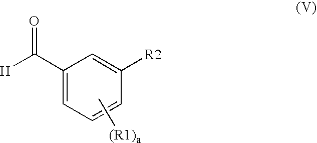
wherein a, R1, and R2 have the same meanings as for formula (I), and thereafter optionally for either process (a) or (b):
removing any protecting groups and/or
converting a compound of formula (I) into another compound of formula (I) and/or
forming a pharmaceutically acceptable salt.

8. A pharmaceutical composition comprising a compound as defined in any of claims 1-6, and a pharmaceutically acceptable diluent, carrier and/or excipient.

9. A process for preparing a composition as defined in claim 8, the process comprising mixing a compound as defined in claim 1 with a pharmaceutically acceptable diluent, carrier and/or excipient.

10. A compound or a composition as defined in claim 1 for use in therapy.

11. A compound or a composition as defined in claim 1 for use in the treatment of a CNS disorder.

12. A compound or a composition as defined in claim 11 wherein the CNS disorder is depression or anxiety.

13. Use of a compound or a composition as defined in claim 1 in the manufacture of a medicament for use in the treatment of a CNS disorder.

14. The use as claimed in claim 13, wherein the disorder is depression or anxiety.

15. A method of treating a CNS disorder in mammals including humans, which comprises administering to the sufferer a therapeutically safe and effective amount of a compound or a composition as defined in claim 1.

16. The method as claimed in claim 15, wherein the disorder is depression or anxiety.

17. Use of a compound of formula (la) or a pharmaceutically acceptable salt thereof: 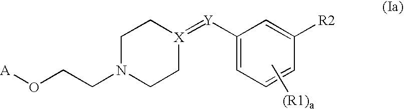
wherein
A is optionally substituted phenyl, indolyl, quinolinyl, quinazolinyl, indazolyl or isoquinolinyl;
X is carbon, Y is CH and ═ is a double bond; or X is CH, Y is CH₂ or oxygen and ═ is a single bond; or X is nitrogen, Y is CH₂ and ═ is a single bond;
R1 is halogen, cyano, nitro, C₁₋₆alkyl, haloC₁₋₆alkyl, C₁₋₆alkoxy, C₃₋₇heterocyclylC₁₋₆alkyl, C₃₋₇heterocyclyl C₁₋₆alkoxy;
d is 0, 1, 2, 3 or 4;
R2 is either:
halogen, —CN, C₃₋₇cycloalkyl, aryl or a C-linked 3-7 membered heterocyclic group;
or
a group -(Z)b-B wherein:
(i) Z is oxygen, CH₂, C═O, SO₂ or C═N—OR3 wherein R3 is hydrogen or C₁₋₆alkyl; b is 1, 2, or 3; and B is hydrogen, C₁₋₆alkyl, C₃₋₇cycloalkyl, C₁₋₆alkoxy or NR4R5 wherein R4 and R5 are independently hydrogen, C₁₋₆alkyl, C₃₋₇cycloalkyl, C₁₋₆alkanoyl, fluoroC₁₋₆alkanoyl, C₁₋₆alkylsulfonyl, fluoroC₁₋₆alkylsulfonyl, carbamoyl or C₁₋₆alkylcarbamoyl, or R4 and R5, together with the nitrogen atom to which they are attached, form part of an optionally substituted 3 to 7 membered heterocyclic group;
or
(ii) Z is oxygen, CH or CH₂, b is 1, and B forms the rest of an aryl or a C₃₋₇heterocyclic group fused to the phenyl ring;
in the manufacture of a medicament for use in the treatment of depression and/or anxiety.

18. The use as claimed in claim 17, wherein, in formula (Ia), A is quinolinyl or quinazolinyl.

19. The use as claimed in 18, wherein A is 5-(2-methyl)quinolinyl or 5-(2-methyl)quinazolinyl.

20. The use as claimed in claim 1, wherein R1 is fluoro.

21. The use as claimed in claim 1, wherein b is 0, 1 or 2.

22. A process for the preparation of a compound of formula (Ia) as defined in claim 1 or a pharmaceutically acceptable salt thereof, which process comprises:
(a) the coupling of a compound of formula (IIa): 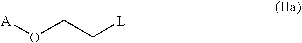
wherein A is as defined for formula (Ia) and L is a leaving group,
with a compound of formula (IIIa): 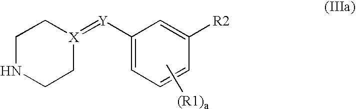
wherein a, Y, R1, R2 and ═ are as defined for formula (Ia);
or
(b) for a compound wherein X is nitrogen, the coupling of a compound of formula (IVa): 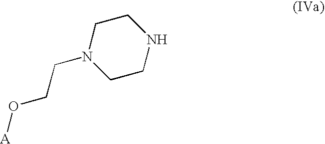
wherein A has the same meaning as for formula (Ia), and a compound of formula (Va): 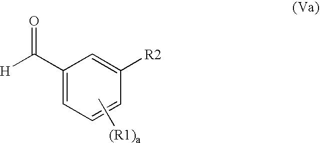
wherein a, R1 and R2 are as defined for formula (Ia),
and thereafter optionally for either process (a) or process (b):
removing any protecting groups and/or
converting a compound of formula (Ia) into another compound of formula (Ia) and/or
forming a pharmaceutically acceptable salt.

23. A method of treating depression or anxiety in mammals including humans, which comprises administering to the sufferer a therapeutically safe and effective amount of a compound of formula (Ia): 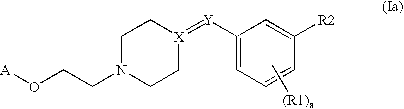
wherein
A is optionally substituted phenyl, indolyl, quinolinyl, quinazolinyl, indazolyl or isoquinolinyl;
X is carbon, Y is CH and is a double bond; or X is CH, Y is CH₂ or, oxygen and ═ is a single bond; or X is nitrogen, Y is CH₂ and ═ is a single bond;
R1 is halogen, cyano, nitro, C₁₋₆alkyl, haloC₁₋₆alkyl, C₁₋₆alkoxy, C₃₋₇heterocyclylC₁₋₆alkyl, C₃₋₇heterocyclyl C₁₋₆alkoxy;
d is 0, 1, 2, 3 or 4;
R2 is either:
halogen, —CN, C₃₋₇cycloalkyl, aryl or a C-linked 3-7 membered heterocyclic group;
or
a group -(Z)b-B wherein:
(i) Z is oxygen, CH₂, C═O, SO₂ or C═N—OR3 wherein R3 is hydrogen or C₁₋₆alkyl; b is 1, 2, or 3; and B is hydrogen, C₁₋₆alkyl, C₃₋₇cycloalkyl, C₁₋₆alkoxy or NR4R5 wherein R4 and R5 are independently hydrogen, C₁₋₆alkyl, C₃₋₇cycloalkyl, C₁₋₆alkanoyl, fluoroC₁₋₆alkanoyl, C₁₋₆alkylsulfonyl, fluoroC₁₋₆alkylsulfonyl, carbamoyl or C₁₋₆alkylcarbamoyl, or R4 and R5, together with the nitrogen atom to which they are attached, form part of an optionally substituted 3 to 7 membered heterocyclic group;
or
(ii) Z is oxygen, CH or CH₂, b is 1, and B forms the rest of an aryl or a C₃₋₇heterocyclic group fused to the phenyl ring.
